(19) United States
(12) Patent Application Publication
(10) Pub. No.: US 2025/0171546 A1
(43) Pub. Date: May 29, 2025

(54) DUPILUMAB FOR TREATMENT OF COVID-19

(71) Applicant: UNIVERSITY OF VIRGINIA PATENT FOUNDATION, Charlottesville, VA (US)

(72) Inventors: William A. Petri, Jr., Charlottesville, VA (US); Alexandra N. Donlan, Seattle, WA (US); Jennifer Sasson, Charlottesville, VA (US)

(73) Assignee: UNIVERSITY OF VIRGINIA PATENT FOUNDATION, Charlottesville, VA (US)

(21) Appl. No.: 18/841,169

(22) PCT Filed: Feb. 24, 2023
(86) PCT No.: PCT/US2023/063279

§ 371 Date: Aug. 23, 2024

Related U.S. Application Data
(60) Provisional application No. 63/313,662, filed on Feb. 24, 2022.

(51) Int. Cl. C07K 16/28; A61K 39/00; A61K 45/06; A61P 31/14

(52) U.S. Cl. CPC C07K 16/2866; A61K 45/06; A61P 31/14; A61K 2039/505

(57) ABSTRACT

Methods for treating coronavirus disease 2019 (COVID-19), the disease caused by severe acute respiratory syndrome coronavirus 2 (SARS-CoV-2) infections, are described. The methods include administration of combinations of agents, including combinations that include dupilumab. The methods can be used to treat subjects diagnosed with a SARS-CoV-2 infection, including those already hospitalized to treat COVID-19 and/or those also having lymphopenia, to reduce the severity of outcomes related to COVID-19, such as admittance to the intensive care unit (ICU), mechanical ventilation, and/or death, particularly over periods of time longer than a month or two months following the initial administration of the agents. Compositions for use in the treatment of COVID-19 are also described.

### CROSS REFERENCE TO RELATED APPLICATIONS

The presently disclosed subject matter claims the benefit of U.S. Provisional Patent Application Ser. No. 63/313,662, filed Feb. 24, 2022; the disclosure of which is incorporated herein by reference in its entirety.

### GOVERNMENT SUPPORT

This invention was made with government support under Grant Nos. R01 AI124214, UL1TR003015, and KL2TR003016 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 102,313 byte UTF-8-encoded XML file created on Feb. 24, 2023 and entitled “3062_180_PCT.xml”. The content of the Sequence Listing XML submitted via Patent Center is incorporated herein by reference in its entirety.

### TECHNICAL FIELD

The presently disclosed subject matter relates generally to methods for treating coronavirus disease 2019 (COVID-19), e.g., to reduce disease severity.

### BACKGROUND

Severe acute respiratory syndrome coronavirus 2 (SARS-CoV-2) is the infectious agent currently causing the global coronavirus disease 2019 (COVID-19) pandemic. SARS-CoV-2 primarily infects the lower respiratory tract of hosts by gaining entry to cells via the receptor angiotensin converting enzyme 2 (ACE2) facilitated by transmembrane receptor neuropilin-1 (Winkler et al., 2020; and Hoffmann et al., 2020). The clinical course following infection varies widely, ranging from asymptomatic carriage to life-threatening respiratory failure and death.

Early on in the pandemic, it was recognized that patients with severe forms of COVID-19, e.g., hospitalization or ventilation, exhibited elevated levels of inflammatory cytokines (Pedersen and Ho, 2020). This inflammatory state can result in end-organ damage and, in some cases, death (Mangalmurti and Hunter, 2020; Tisoncik et al., 2012). In support of this hypothesis, the use of the anti-inflammatory steroid dexamethasone, was found to decrease mortality by 29% in COVID-19 patients requiring mechanical ventilation (The RECOVERY Collaborative Group, 2020).

With continued surges in the COVID-19 pandemic, the probability of achieving herd immunity has significantly decreased, owing to vaccine hesitancy, uneven global vaccine roll out, rapid viral spread, the increasing transmissibility of viral variants and evasion of preventative measures (Aschwanden, 2021). Thus, while mortality rates have improved with experience and resource preparedness, there remains an ongoing need for further development of therapies to combat respiratory failure in those requiring hospitalization (Ritchie et al., 2020). There also remains an ongoing need for additional therapies to combat other severe clinical outcomes, e.g., to reduce intensive care unit (ICU) admittance among those hospitalized with COVID-19, to reduce the length of hospitalization, to reduce ventilation risk, and to reduce mortality.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of treating coronavirus disease 2019 (COVID-19) in a human subject in need thereof, wherein the method comprises administering to the subject at least two of: (i) one or more of a steroid, an antiviral agent, baricitinib, an interleukin-6 inhibitor, and an anti-severe acute respiratory syndrome coronavirus 2 (SARS-CoV-2) monoclonal antibody; (ii) a composition comprising dupilumab or a Fab, F(ab′)₂, or scFv fragment thereof; and (iii) a composition comprising a chitinase-3-like protein 1 (YKL-40) blockade agent, a composition comprising a monocyte chemoattractant protein 1 (MCP-1) blockade agent, a composition comprising a hyaluronan blockade agent, or any combination thereof. In some embodiments, the method comprises administering (ii) in combination with either (i) or (iii). In some embodiments, the method comprises administering (iii) in combination with either (i) or (ii). In some embodiments, the method comprises administering (i), (ii), and (iii).

In some embodiments, the human subject is a human subject who has tested positive for a SARS-CoV-2 infection within the last 14 days and/or is hospitalized for the treatment of COVID-19. In some embodiments, the human subject shows evidence of lower respiratory disease during clinical assessment or imaging. In some embodiments, the human subject has one or more symptom of the group comprising a saturation of oxygen (SpO₂) less than about 94% on room air at sea level, lung infiltrates of greater than about 50%, a respiratory frequency of more than about 30 breaths per minute, and a ratio of arterial partial pressure of oxygen to fraction of inspired oxygen (PaO₂/FiO₂) of less than about 300 millimeters of mercury (mmHg).

In some embodiments, the human subject is a human subject with lymphopenia.

In some embodiments, the administering reduces the risk that the human subject is admitted to an intensive care unit (ICU), optionally wherein the administering reduces the risk that the human subject is admitted to an ICU for the treatment of COVID-19 within a time period of about 60 days or more following the administering. In some embodiments, the administering reduces the risk of death, optionally wherein the administering reduces the risk of death due to COVID-19 for a time period of about 60 days or more following the administering.

In some embodiments, the administering of the composition comprising dupilumab or a Fab, F(ab′)2, or scFv fragment thereof is performed subcutaneously. In some embodiments, the administering comprises administering two or more doses of the composition that comprises dupilumab or a Fab, F(ab′)2, or scFv fragment thereof, optionally wherein said two more doses are administered about 14 days apart.

In some embodiments, the administering reduces a plasma level of YKL-40 and/or MCP-1. In some embodiments, the administering reduces hyaluronan synthesis.

In some embodiments, the presently disclosed subject matter provides a method of treating COVID-19 in a human subject in need thereof, wherein the method comprises administering to the subject a composition comprising a YKL-40 blockade agent and/or a composition comprising a MCP-1 blockade agent. In some embodiments, the YKL-40 blockade agent and/or the MCP-1 blockade agent is a monoclonal antibody.

In some embodiments, the presently disclosed subject matter provides a composition for use in the treatment of COVID-19 in a human subject in need thereof, wherein the composition comprises at least two of: (i) one or more of a steroid, an antiviral agent, baricitinib, an interleukin-6 inhibitor, and an anti-SARS-CoV-2 monoclonal antibody; (ii) a composition comprising dupilumab or a Fab, F(ab′)2, or scFv fragment thereof, and (iii) a composition comprising a YKL-40 blockade agent, a composition comprising a MCP-1 blockade agent, a composition comprising a hyaluronan blockade agent, or any combination thereof. In some embodiments, the composition comprises (ii) and either (i) or (iii). In some embodiments, the composition comprises (iii) and either (i) or (ii). In some embodiments, the composition comprises (i), (ii), and (iii).

In some embodiments, the human subject is a human subject who has tested positive for a SARS-CoV-2 infection within the last 14 days and/or is hospitalized for the treatment of COVID-19. In some embodiments, the human subject shows evidence of lower respiratory disease during clinical assessment or imaging. In some embodiments, the human subject has one or more symptom of the group comprising a saturation of oxygen (SpO₂) less than about 94% on room air at sea level, lung infiltrates of greater than about 50%, a respiratory frequency of more than about 30 breaths per minute, and a ratio of arterial partial pressure of oxygen to fraction of inspired oxygen (PaO₂/FiO₂) of less than about 300 millimeters of mercury (mmHg).

In some embodiments, the human subject is a human subject having lymphopenia.

In some embodiments, the presently disclosed subject matter provides a composition for use in the treatment of COVID-19 in a human subject in need thereof, wherein the composition comprises a YKL-40 blockade agent and/or a composition comprising a MCP-1 blockade agent. In some embodiments, the composition comprises a YKL-40 blockade agent and/or a MCP-1 blockade agent, optionally wherein the YKL-40 blockade agent and/or the MCP-1 blockade agent is a monoclonal antibody.

Accordingly, it is an object of the presently disclosed subject matter to provide methods of treating COVID-19 and compositions for use therein.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and Examples. Additionally, various aspects and embodiments of the presently disclosed subject matter are described in further detail below.

### BRIEF DESCRIPTION OF THE FIGURES

FIG. 1 is a schematic drawing showing the timeline of a randomized, double blind, placebo-controlled trial used to study longer term effects of treating patients with moderate to severe coronavirus disease 2019 (COVID-19) with dupilumab.

FIG. 2 is a graph showing Kaplan-Meier curves depicting the sixty (60)-day mortality between the two treatment groups (dupilumab, thinner gray line and placebo, heavier gray line) from the trial described in FIG. 1.

FIG. 3 is a graph showing Kaplan-Meier curves depicting need for escalation to the intensive care unit (ICU) over the initial sixty (60)-day study period in the patients in the dupilumab (thinner gray line) and placebo (heavier gray line) groups from the trial described in FIG. 1.

FIG. 4 is a graph showing the Kaplan-Meier curves depicting 60-day mortality between two treatment groups from the trial described in FIG. 1 in only those patients identified with lymphopenia (defined as a lymphocyte count of less than 1000 per microliter (<1.0 K/μl)) on study day 0. The x-axis shows survival time (SURVTIME) in days. Dupilumab group represented by thinner gray line. Placebo group represented by heavier gray line.

FIGS. 5A and 5B are graphs showing the Kaplan-Meier curves depicting three hundred and sixty (360)-day mortality in the two treatment groups described in in the trial described in FIG. 1. FIG. 5A is a graph of the Kaplan-Meier curves for all of the patients, while FIG. 5B is a graph of the Kaplan-Meier curves for the sub-group of patients with lymphopenia on study day 0. Dupilumab group represented by thinner gray line. Placebo group represented by heavier gray line. The x-axis of each graph shows survival time (SURVTIME_360) in days.

FIG. 6 shows a pair of graphs of the National Institute of Allergy and Infectious Disease (NIAID) ordinal scores over time (in days) by treatment group from patients in the trial as described in FIG. 1. The proportion of patient ordinal scores at designated time points throughout the initial sixty days of the study period are split into dupilumab (left) and placebo (right) groups. The group percentage for the ordinal score is listed within the box for each score. The ordinal scale is as followed: 8) Death; 7) Hospitalized, on invasive mechanical ventilation or extracorporeal membrane oxygenation (ECMO); 6) Hospitalized, on non-invasive ventilation or high flow oxygen devices; 5) Hospitalized, requiring supplemental oxygen; 4) Hospitalized, not requiring supplemental oxygen—requiring ongoing medical care (COVID-19 related or otherwise); 3) Hospitalized, not requiring supplemental oxygen—no longer requires ongoing medical care; 2) Not hospitalized, limitation on activities and/or requiring home oxygen; 1) Not hospitalized, no limitations on activities.

FIG. 7 is a series of graphs of the saturated oxygen versus the fraction of inspired oxygen (SaO₂/FiO₂) ratio or the arterial partial pressure of oxygen versus the fraction of inspired oxygen (PaO₂/FiO₂) ratio over time by treatment group and oxygenation strategy. Median ratios at each time point are designated as the horizontal line within the boxes. Mean ratios are depicted as diamonds within the boxes. The dupilumab treatment group is depicted in boxes on the left and placebo groups on the right. PaO₂/FiO₂ ratios over time shown in the top boxes for those requiring mechanical ventilation. SaO₂/FiO₂ ratio over time shown in the bottom boxes for those requiring non-invasive oxygenation measures.

FIG. 8 is a graph of C-reactive protein (CRP, value shown as the natural log (Ln) transformed of concentration in milligrams per deciliter (mg/dL)) level on study day 0 (represented by boxes on the left for each treatment group) and study day 14 (represented by boxes on the right for each treatment group) by treatment group (dupilumab, left; placebo, right). The horizontal dotted line is indicative of the lower limit of normal for measurement via clinical lab. The solid horizontal line within each box is representative of the median value and the open circle within each box is representative of the mean value. Open circles outside of a box represent outliers.

FIG. 9 is a graph of the ferritin (value shown as the natural log (Ln) transformed of concentration in nanograms per milliliter (ng/mL)) level on study day 0 (represented by boxes on the left for each treatment group) and study day 14 (represented by boxes on the right for each treatment group) by treatment group (dupilumab, left; placebo, right). The horizontal dotted line is indicative of the lower limit of normal for measurement via clinical lab. The solid horizontal line within each box is representative of the median value and the open circle within each box is representative of the mean value. The open circle outside of a box represents an outlier.

FIG. 10 is a graph of immunoglobulin E (IgE, natural log (ln) transformed for IgE measured in international units per milliliter (IU/mL)) level on study day 0 (represented by boxes on the left of each pair) and study day 14 (represented by boxes on the right of each pair) by treatment group. The horizontal dotted line is indicative of the lower limit of normal for measurement via clinical lab. The solid horizontal line within each box is representative of the median value and the open circle within each box is representative of the mean value. The open circle outside of a box represents an outlier.

FIGS. 11A-11C are a series of graphs showing the levels of: (FIG. 11A) Absolute lymphocytes (thousands per microliter (K/UL)), (FIG. 11B) neutrophils (thousands per microliter (K/μL)) and (FIG. 11C) eosinophils (thousands per microliter (K/μL)) over time (in days) for each treatment group. Median*** count with standard error bars are depicted at study days 0, 2, 5, 7, 14 split between dupilumab and placebo groups. The solid horizontal lines within each box are representative of the median value and the open circles within each box are representative of the mean value. The open circles outside of boxes represent outliers.

FIGS. 12A-12C are a series of graphs showing box and whisker plots of (FIG. 12A) chitinase-3-like protein 1 (YKL-40, in picograms per milliliter (pg/ml)), (FIG. 12B) eotaxin 3 (in pg/ml), and (FIG. 12C) monocyte chemoattractant protein 1 (MCP-1, in pg/ml) in patient serum at various time points throughout the study period. The X axis represents the study day when the serum sample was taken. The boxes to the right of each pair represent the dupilumab treatment group. The boxes to the left of each pair represent the placebo treatment group.

FIG. 13 is a graph showing the Kaplan-Meier curves for 60-day survival probability by treatment group and N-protein (NP) quantile. Subjects in the dupilumab arm whose baseline N-protein level was in the top 25^{th} percentile of the group are represented by the thinner dashed gray line. Subjects in the placebo arm whose baseline N-protein level was in the top 25^{th} percentile of the group are represented by the heavier dashed gray line. Subjects in the dupilumab and placebo arms in the rest of the cohort (i.e., bottom 75^{th} percentile) are represented by the thinner and heavier solid gray lines, respectively.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a therapeutic agent” refers to one or more therapeutic agents, e.g., one or more of the same of different therapeutic agents. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of time, concentration percent inhibition, percent viability, amounts of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” unless stated otherwise.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the presently disclosed subject matter can exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “secondary antibody” refers to an antibody that binds to the constant region of another antibody (the primary antibody).

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen can induce the production of antibodies that bind specifically to a given region or three-dimensional structure on the protein; these regions or structures are referred to as antigenic determinants. An antigenic determinant can compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “blockade agent” refers to an agent that blocks and/or neutralizes and/or reduces the activity of a named target entity (such as but not limited to by binding, including selectively binding, to and/or degrading said named target entity (or a binding partner thereof) or by reducing the synthesis of the named target entity. Thus, in some embodiments, the term “blockade agent” as used herein refers to an agent that binds to the named “blockaded” or “neutralized” entity or a biological binding partner (e.g., receptor) thereof, thereby decreasing or inhibiting binding of the named neutralized entity and its binding partner and/or otherwise decreasing or inhibiting one or more biological activity of the named blockaded entity. An exemplary hyaluronan blockade agent, i.e., 4-methylumbelliferone (4-MU), for instance, is a substrate for an enzyme in HA synthesis.

As used herein, the term “conservative amino acid substitution” is defined herein as an amino acid exchange within one of the following five groups: I) Small aliphatic, nonpolar or slightly polar residues: Ala, Ser, Thr, Pro, Gly; II) Polar, negatively charged residues and their amides: Asp, Asn, Glu, Gln; III) Polar, positively charged residues: His, Arg, Lys; IV) Large, aliphatic, nonpolar residues: Met Leu, Ile, Val, Cys; and V) Large, aromatic residues: Phe, Tyr, Trp.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

“Co-administer” can include simultaneous and/or sequential administration of two or more agents.

A “compound,” as used herein, refers to any type of substance or agent that is can be considered a drug, or a candidate for use as a drug, as well as combinations and mixtures of the above.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, a “derivative” of a compound refers to a chemical compound that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment” or “segment” or “subsequence” is a portion of an amino acid sequence comprising at least one amino acid or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, can ordinarily be at least about 20 nucleotides in length, typically, at least about 50 nucleotides, more typically, from about 50 to about 100 nucleotides, at least about 100 to about 200 nucleotides, at least about 200 nucleotides to about 300 nucleotides, at least about 300 to about 350, at least about 350 nucleotides to about 500 nucleotides, at least about 500 to about 600, at least about 600 nucleotides to about 620 nucleotides, at least about 620 to about 650, and or the nucleic acid fragment will be greater than about 650 nucleotides in length.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm incorporated into the NBLAST and XBLAST programs that can be accessed, for example, at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized. Alternatively, PSI-Blast or PHI-Blast can be used to perform an iterated search which detects distant relationships between molecules and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein “injecting”, “applying”, and “administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample can of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent” as used herein means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein. In some embodiments, the subject is a human subject.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with the each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from a method of the presently disclosed subject matter.

The term “substantially pure” describes a compound, e.g., a protein or polypeptide, cell or nucleic acid that has been separated from components which naturally accompany it. Typically, a compound is substantially pure when at least 10%, including at least 20%, at least 50%, at least 60%, at least 75%, at least 90%, at least 95%, at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, a “substantially homologous amino acid sequences” or “substantially identical amino acid sequences” includes those amino acid sequences which have at least about 92%, or at least about 95% homology or identity, including at least about 96% homology or identity, including at least about 97% homology or identity, including at least about 98% homology or identity, and at least about 99% or more homology or identity to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” or “substantially identical nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In one embodiment, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 92%, 95%, 99% or more. Substantial identity of nucleic acid sequences can be determined by comparing the sequence identity of two sequences, for example by physical/chemical methods (i.e., hybridization) or by sequence alignment via computer algorithm.

Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C., with washing in 1×SSC, 0.1% SDS at 50° C.; preferably 7% SDS, 0.5 M NaPO₄, 1 mM EDTA at 50° C. with washing in 0.5 SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package. The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing or eliminating, that results in the improvement of the condition, disease, disorder, and the like, or ameliorating a symptom thereof.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. General Considerations

Despite advances, a profound need remains to develop further therapeutics for treatment of those hospitalized with COVID-19 to prevent respiratory decline, mechanical ventilation, and death. Studies described herein assess the safety and efficacy of dupilumab, a monoclonal antibody which blocks interleukin-13 (IL-13) and interleukin-4 (IL-4) signaling, for use in those hospitalized with COVID-19, and the impact of type 2 immune blockade on progression of disease.

Dupilumab, an anti-IL-4Rα monoclonal antibody, has been FDA approved for treatment of moderate to severe atopic dermatitis since 2017. It has been successfully shown to reduce disease severity in atopic dermatitis and other allergic diseases where type 2 cytokines have been implicated, including asthma and chronic rhinosinusitis (Thibodeaux et al., 2019). Original clinical trials noted limited adverse events favoring its use for treatment of atopic patients as a steroid sparing regimen (Wenzel et al., 2016; Blauvelt et al., 2017). Dupilumab is supplied as a single-dose pre-filled syringe, is administered subcutaneously, and reaches peak mean concentrations within one-week post dose (FDA, 2020).

IL-13 is associated with poor outcomes in COVID-19 disease in humans and neutralization of IL-13 prevents death in a mouse model of SARS-CoV-2 infection (Donlan et al., 2021). IL-13, which co-recognizes the receptor IL4Rα along with interleukin-4 (IL-4), is a cytokine of the type 2 immune pathway previously known to be involved in eosinophilic inflammation, mucous secretion, goblet cell metaplasia, and fibrosis, and has been regularly implicated in airway hyperresponsiveness and atopic disease (Wynn, 2003). Utilizing a biorepository of patient COVID-19 plasma samples, it was discovered that patients with IL-13 levels in the highest quantile had a significantly greater risk of needing mechanical ventilation than those in the lowest quantile (Donlan et al., 2021). See also PCT International Publication No. WO 2021/257888, the disclosure of which is incorporated herein by reference in its entirety. In addition, the potential role of IL-13 in progression to severe illness from COVID-19 using a large international database called TriNETX. A cohort of 350,004 cases of COVID-19 were identified, in which 81 patients had been prescribed dupilumab independent of their COVID-19 status (Donlan et al., 2021). Patients on dupilumab were 1:1 propensity scored matched to a subcohort of 81 patients with COVID-19 with atopic diseases for which dupilumab is routinely used. There were zero deaths found in the patients on dupilumab, and these patients had a 12.5% risk reduction in mortality compared to those not on dupilumab.

As described hereinbelow, a phase IIa randomized double-blind placebo-controlled trial was conducted to assess the safety and efficacy of dupilumab plus standard of care versus placebo plus standard of care in mitigating respiratory failure and death in those hospitalized with COVID-19. Forty eligible subjects were enrolled and randomized to receive either dupilumab or placebo. Subjects were followed for up to 360 days, with collection of clinical endpoints, adverse events and immunologic biomarkers at multiple time points throughout the study period. The primary endpoint was the proportion of patients alive and free of invasive mechanical ventilation at 28 days, assessed via time to event analysis.

There was no difference in adverse events detected between the two treatment groups. There was also no significant difference in proportion of patients alive and free of mechanical ventilation at day 28 between study treatment groups. However, at day 60, 89.5% subjects who had received dupilumab were alive and free of mechanical ventilation compared to 76.2% for those who had received placebo (log rank p=0.25, adjusted p=0.026 adjusting for gender, ventilation and vaccination status in Cox regression). Furthermore, 52.4% of patients who received placebo required escalation to the ICU compared to 21.1% of patients who received dupilumab (Fisher's exact p=0.06). After adjusting for gender and vaccination status in logistic regression, those who received placebo appeared more likely to require escalation to the ICU during their admission compared to those who received dupilumab (OR 0.31, CI: 0.07-1.3, p=0.11). Lastly, adjusting for dynamic mechanical ventilation status, dupilumab significantly reduced mortality at day 60 compared to placebo (HR 0.06, 95% CI: 0.01-0.71, p=0.03, p=0.03 when adjusting for gender and vaccination status).

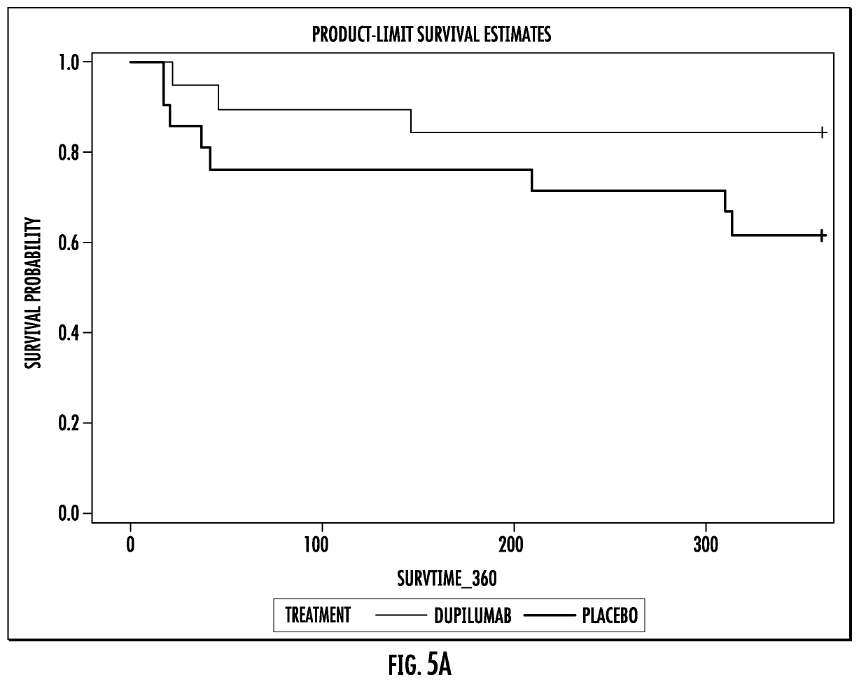

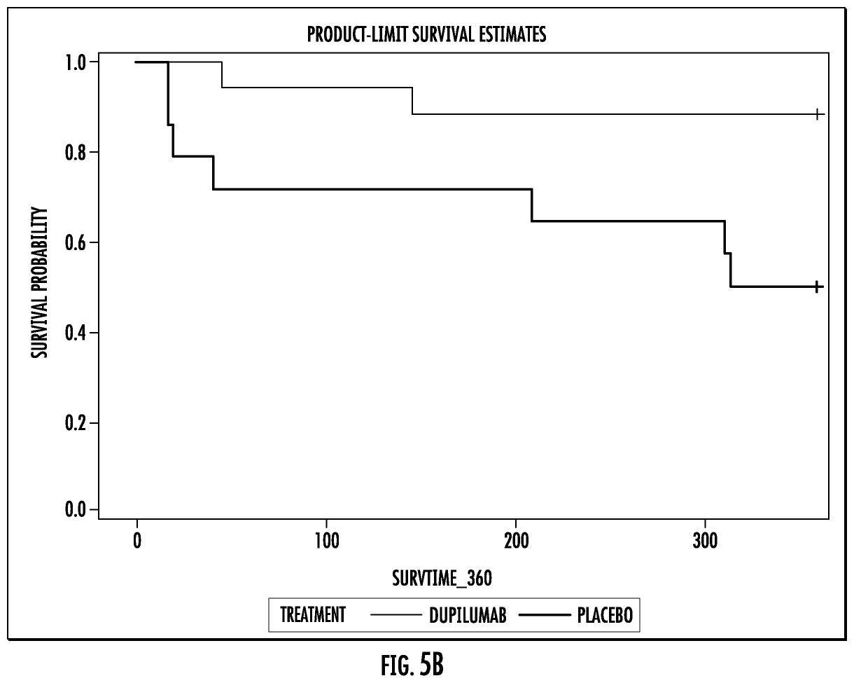

By day 360, the mortality reduction in subjects receiving dupilumab was still observed (FIG. 5A). Patients with lymphopenia at the time of randomization showed a greater survival benefit with dupilumab vs placebo, compared to all subjects in the phase 2 study (FIG. 5B).

### III. Methods of and Compositions for Treating Covid-19

In some embodiments, the presently disclosed subject matter provides a method of treating COVID-19 in a subject in need thereof, e.g., a human subject in need thereof, e.g., a human subject suffering from and/or diagnosed with a SARS-CoV-2 infection. In some embodiments, the method can include administration of a combination of at least two therapeutic agents, wherein the at least two therapeutic agents include at least one agent selected from at least two of groups (i)-(iii), where groups (i)-(iii) are as follows: (i) therapeutic agents included in “standard of care” treatment agents for COVID-19, such as, but not limited to, a steroid, an antiviral agent, baricitinib, an interleukin-6 (IL-6) inhibitor, and an anti-SARS-CoV-2 monoclonal antibody (mAb); (ii) dupilumab or a fragment thereof that can bind the same antigen as dupilumab, such as a fragment that comprises an antigen binding region or variable chains thereof (e.g., a monovalent antigen-binding region fragment (i.e., a Fab), a divalent antigen-binding region fragment (i.e., a F(ab)₂), or a single chain variable fragment (scFv) of dupilumab); and (iii) blockade agents of chitinase-3-like protein 1 (YKL-40), monocyte chemoattractant protein 1 (MCP-1) and hyaluronan. Thus, in some embodiments, the method comprises administering (i.e., sequentially or simultaneously co-administering) to a subject in need thereof (e.g., a human subject in need thereof), at least two of: (i) one or more of the group comprising, consisting essentially of, or consisting of a steroid (e.g., dexamethasone or prednisone), an antiviral agent (e.g., remdesivir), baricitinib, an IL-6 inhibitor, and an anti-SARS-CoV-2 mAb; (ii) a composition comprising dupilumab or a Fab, F(ab′)₂, or scFv thereof; and (iii) a composition comprising a YKL-40 blockade agent, a composition comprising a MCP-1 blockade agent, a composition comprising a hyaluronan blockade agent, or any combination thereof. In some embodiments, the method comprises administering to the subject: (i) one or more of the group comprising, consisting essentially of, or consisting of a steroid (e.g., dexamethasone or prednisone), an antiviral agent (e.g., remdesivir), baricitinib, an interleukin-6 inhibitor, and an anti-severe acute respiratory syndrome coronavirus 2 (SARS-CoV-2) monoclonal antibody; (ii) a composition comprising dupilumab or a Fab, F(ab′)₂, or scFv fragment thereof, and (iii) a composition comprising a YKL-40 blockade agent, a composition comprising a MCP-1 blockade agent, a composition comprising a hyaluronan blockade agent, or any combination thereof.

In some embodiments, the method comprises administering (ii), i.e., a composition comprising dupilumab or a Fab, F(ab′)2, or scFv thereof, in combination with either (i) or (iii). For example, in some embodiments, the method comprises administering: (i) one or more of the group comprising, consisting essentially of, or consisting of a steroid (e.g., dexamethasone or prednisone), an antiviral agent (e.g., remdesivir), baricitinib, an interleukin-6 inhibitor, and an anti-SARS-CoV-2 mAb; and (ii) a composition comprising dupilumab or a Fab, F(ab′)2, or scFv thereof. In some embodiments, the presently disclosed subject matter provides a method for treating COVID-19 in a subject in need thereof (e.g., a human subject in need thereof, such as a subject suffering from or diagnosed with a SARS-CoV-2 infection), wherein the method comprises administering to the subject: (i) one or more of the group comprising, consisting essentially of, or consisting of a steroid (e.g., dexamethasone or prednisone), remdesivir, baricitinib, an interleukin-6 inhibitor, and an anti-severe acute respiratory syndrome coronavirus 2 (SARS-CoV-2) monoclonal antibody; and (ii) a composition comprising dupilumab or a Fab, F(ab′)₂, or scFv fragment thereof.

Dupilumab is a mAb (sold under the tradename DUPIXENT®, Sanofi Biotechnology, Paris, France), developed by Sanofi Genzyme (Cambridge, Massachusetts, United States of America) and Regeneron Pharmaceuticals (Tarrytown, New York, United States of America). The amino acid sequences for the heavy and light chains of dupilumab are:

EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
G
(Dupilumab heavy chain; SEQ ID NO: 38)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(Dupilumab light chain; SEQ ID NO: 39)

Dupilumab is approved by the FDA for use in treating moderate to severe atopic dermatitis and asthma. It is an anti-human interleukin-4 receptor-alpha (IL-4Rα) antibody that can block the interleukin-4 (IL-4)/IL4Rα signaling pathway. The IL-4/IL4Rα complex can recruit either γc to form the Type I IL-4 receptor or interleukin-13 receptor alpha 1 (IL-13Rα1) to form the Type II IL-4 receptor.

Dupilumab or the fragment thereof can be administered using similar dosage routes and/or doses to those used with dupilumab in treating other diseases. For instance, in some embodiments, dupilumab can be administered according to the presently disclosed method via routes and using dosages similar to those used when dupilumab is used to treat atopic dermatitis, e.g., via sub-cutaneous injection using a loading dose of 600 milligrams (as two 300 mg doses), with additional 300 mg doses every two weeks, as needed. Thus, in some embodiments, the administering of (ii), i.e., the composition comprising dupilumab or a Fab, F(ab′)2, or scFv fragment thereof, is performed subcutaneously. In some embodiments, the method comprises administering two or more doses (e.g., two doses or three doses) of the composition that comprises dupilumab or a Fab, F(ab′)2, or scFv fragment thereof. In some embodiments, the two more doses are administered about 14 days apart.

The one or more agent of group (i), i.e., the one or more of a steroid (e.g., a corticosteroid, such as dexamethasone or prednisone), an antiviral agent (e.g., remdesivir), baricitinib, a IL-6 inhibitor, or an anti-SARS-CoV-2 mAb can be administered in dosages and via routes known in the art, either in the treatment of COVID-19 or another disease for which the standard of care agent is used. Dexamethasone, for example, can be administered orally, intravenously or via infusion. Remdesivir is sold under the tradename VEKLURY® (Gilead Sciences, Cork, Ireland). In addition to remdesivir, other antiviral agents that have been used to treat COVID-19 include molnupiravir (sold under the tradename LAGEVRIO®, Merck Sharpe & Dohme LLC, Rahway, New Jersey, United States of America) and the combination of nirmatrelvir co-packaged with ritonavir (sold under the tradename PAXLOVID®, Pfizer, Inc., New York, New York, United States of America). Baricitinib (sold under the tradename OLUMIANT®; Eli Lilly and Company, Indianapolis, Indiana, United States of America), was originally approved for use in treating rheumatoid arthritis (RA) and is an orally available small molecule that is used for treating COVID-19 (Mogul et al., 2019).

Suitable IL-6 inhibitors that can be used include antibodies (e.g., mAbs) that bind to IL-6 or the IL-6 receptor. More particularly, in some embodiments, the IL-6 inhibitors can include tocilizumab, sarilumab, and siltuximab. Tocilizumab (sold under the brand name ACTEMRA®; Chugai Seiyabu Kabushiki Kaisha Corporation, Tokyo, Japan) and sarilumab (sold under the brand name KEVZARA®, Sanofi Biotechnology Corporation, Paris, France) both target the IL-6 receptor, while siltuximab (sold under the brand name SYLVANT®, Eusa Pharma (UK) Limited, Hempstead, United Kingdom) binds to IL-6 itself. Amino acid sequences for the heavy and light chains of sarilumab and siltuximab are:

EVQLVESGGGLVQPGRSLRLSCAASRFTFDDYAMHWVRQAPGKGLEWVSG
ISWNSGRIGYADSVKGRFTISRDNAENSLFLQMNGLRAEDTALYYCAKGR
DSFDIWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVTYL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
(sarilumab heavy chain; SEQ ID NO: 40)
DIQMTQSPSSVSASVGDRVTITCRASQGISSWLAWYQQKPGKAPKLLIYG
ASSLESGVPSRFSGSGSGTDFTLTISSLQPEDFASYYCQQANSFPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(sarilumab light chain; SEQ ID NO: 41)
EVQLVESGGKLLKPGGSLKLSCAASGFTFSSFAMSWFRQSPEKRLEWVAE
ISSGGSYTYYPDTVTGRFTISRDNAKNTLYLEMSSLRSEDTAMYYCARGL
WGYYALDYWGQGTSVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
(siltuximab heavy chain; SEQ ID NO: 42)
QIVLIQSPAIMSASPGEKVTMTCSASSSVSYMYWYQQKPGSSPRLLIYDT
SNLASGVPVRFSGSGSGTSYSLTISRMEAEDAATYYCQQWSGYPYTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
(siltuximab light chain; SEQ ID NO: 42)

In some embodiments, the IL-6 inhibitor is tocilizumab.

Anti-SARS-CoV-2 mAbs that have been used to treat COVID-19 include, for example, bebtelovimab (available from Eli Lilly and Company, Indianapolis, Indiana, United States of America) and the combination of two antibodies, tixagevimab and cilgavimab, co-packaged and sold under the tradename EVUSHELD® (AstraZeneca, Cambridge, United Kingdom). These antibodies bind to the spike protein of human SARS-CoV-2. Other antibodies that have been used to treat COVID-19 include, but are not limited to bamlanivimab, etesevimab, the combination of casirivimab and imdevimab (i.e., sold under the tradename REGEN-COV®, Regeneron Pharmaceuticals, Inc., Tarrytown, New York, United States of America), and sotrovimab. The heavy and light chain amino acid sequences of bebtelovimab, tixagevimab, and cilgavimab are:

QITLKESGPTLVKPTQTLTLTCTFSGFSLSISGVGVGWLRQPPGKALEWL
ALIYWDDDKRYSPSLKSRLTISKDTSKNQVVLKMTNIDPVDTATYYCAHH
SISTIFDHWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(bebtelovimab heavy chain; SEQ ID NO: 44)
QSALTQPASVSGSPGQSITISCTATSSDVGDYNYVSWYQQHPGKAPKLMI
FEVSDRPSGISNRFSGSKSGNTASLTISGLQAEDEADYYCSSYTTSSAVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS
(bebtelovimab light chain; SEQ ID NO: 45)
QMQLVQSGPEVKKPGTSVKVSCKASGFTFMSSAVQWVRQARGQRLEWIGW
IVIGSGNTNYAQKFQERVTITRDMSTSTAYMELSSLRSEDTAVYYCAAPY
CSSISCNDGFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEFEGGPSVFLFP
PKPKDTLYITREPEVTCVVVDVSHEDPEVKFNWYVDGVEVHNAKTKPREE
QYNSTYRVVSVLTVLHQDWLNGKEYKCKVSNKALPASIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
(tixagevimab heavy chain; SEQ ID NO: 46)
EIVLTQSPGTLSLSPGERATLSCRASQSVSSSYLAWYQQKPGQAPRLLIY
GASSRATGIPDRFSGSGSGTDFTLTISRLEPEDFAVYYCQHYGSSRGWTF
GQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
(tixagevimab light chain; SEQ ID NO: 47)
EVQLVESGGGLVKPGGSLRLSCAASGFTFRDVWMSWVRQAPGKGLEWVGR
IKSKIDGGTTDYAAPVKGRFTISRDDSKNTLYLQMNSLKTEDTAVYYCTT
AGSYYYDTVGPGLPEGKFDYWGQGTLVTVSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPEFEG
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPASIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
(cilgavimab heavy chain; SEQ ID NO: 48)
DIVMTQSPDSLAVSLGERATINCKSSQSVLYSSNNKNYLAWYQQKPGQPP
KLLMYWASTRESGVPDRFSGSGSGAEFTLTISSLQAEDVAIYYCQQYYST
LTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(cilgavimab light chain; SEQ ID NO: 49)

With the passage of time, it can be expected that additional or alternative anti-SARS-CoV-2 antibodies, i.e., that target a spike protein or the nucleocapsid protein of SARS-CoV-2 or variants of SARS-CoV-2, will be added to the list of FDA approved COVID-19 treatments, depending upon the frequency of variants circulating in a target population at a particular time. In addition, in some embodiments, group (i) can further include anakinra, an anti-interleukin 1 (IL-1) receptor agonist sold under the tradename KINERET® (Swedish Orphan Biovitrium AB, Solna, Sweden).

In some embodiments, the one or more agents recited for (i) comprise two or more of the agents recited for (i), which can include two or more of the same class of agents (e.g., two or more antiviral agents) or two or more agents wherein at least two agents are from different classes (e.g., a steroid and an antiviral agent or one or more antiviral agents and an anti-SARS-CoV-2 antibody). In some embodiments, (i) comprises, consists essentially of, or consists of one or more of a steroid (e.g., dexamethasone or prednisone), an antiviral agent (e.g., remdesivir), tocilizumab, bebtelovimab, tixagevimab, and cilgavimab. In some embodiments, (i) comprises, consists essentially of, or consists of one or more of a steroid and remdesivir. Thus, in some embodiments, the method comprises administering remdesivir and dupilumab; a steroid and dupilumab; or a steroid, remdesivir, and dupilumab.

In some embodiments, the presently disclosed method comprises administering (iii), i.e., a composition comprising a YKL-40 blockade agent, a composition comprising a MCP-1 blockade agent, a composition comprising a hyaluronan blockade agent, or any combination thereof, in combination with either (i) or (ii). As used herein, the term “blockade agent” refers to an agent that blocks and/or neutralizes and/or reduces the activity of a named target entity (such as but not limited to by binding, including selectively binding, to and/or degrading said named target entity (or a binding partner thereof) or by reducing the synthesis of the named target entity. Thus, in some embodiments, the term “blockade agent” as used herein refers to an agent that binds to the named “blockaded” or “neutralized” entity or a biological binding partner (e.g., receptor) thereof, thereby decreasing or inhibiting binding of the named neutralized entity and its binding partner and/or otherwise decreasing or inhibiting one or more biological activity of the named blockaded entity (e.g., YKL-40, MCP-1, or hyaluronan). In some embodiments, the blockage can result in a decrease in a biological activity by at least about 10%, about 20%, about 30%, about 40%, about 50%, about 60%, about 70%, about 80%, about 90%, or about 95% or more, as compared to the level of biological activity of the named entity in the absence of the blockade agent. Representative YKL-40, MCP-1, and hyaluronan blockade agents include but are not limited to small molecule inhibitors, proteins (including binding proteins and enzymes, peptides, nucleic acids, and antibodies and antibody fragments (e.g., Fabs, F(ab′)₂s, and scFvs). The YKL-40 blockade agent, the MCP-1 blockage agent and the hyaluronan blockade agent of (iii) is an agent other than dupilumab or a fragment thereof. In some embodiments, administering (iii) comprises administering a YKL-40 blockade agent or a MCP-1 blockage agent. In some embodiments, administering (iii) comprises administering a YKL-40 blockade agent.

Chitinase-3-like protein 1 (YKL-40 or CHI3L1) is a glycoprotein associated with certain cancers and other pathogenic conditions (e.g., inflammation). YKL-40 blockade agents include, for example, small molecule YKL-40 inhibitors, antisense molecules, and anti-YKL-40 antibodies (e.g., mAbs) or fragments thereof (e.g., Fab, Fab′, F(ab′)2 or scFV fragments). Anti-YKL-40 mAbs have been previous described, e.g., for use in neutralizing YKL-40 in the treatment certain cancers, such as ovarian cancer or brain tumors or in the treatment of nonalcoholic fatty liver disease (U.S. Pat. No. 11,155,638, the disclosure of which is incorporated herein by reference in its entirety; PCT International Publication No. WO 2021/013884, the disclosure of which is incorporated herein by reference in its entirety; Salamon et al., 2014; Faibish et al, 2011; Kang et al., 2020). Anti-YKL-40 antibodies are commercially available, for example, from Invitrogen (Waltham, Massachusetts, United States of America) and Abcam (Cambridge, United Kingdom). Additional anti-YKL-40 antibodies can be prepared via techniques known in the art using a YKL-40 protein or antigentic fragment thereof as an antigen. The sequence of human chitinase-3-like protein 1 precursor (Uniprot No. P36222) is:

mgvkasqtgf vvlvllqccs ayklvcyyts wsqyregdgs
cfpdaldrfl cthiiysfan isndhidtwe wndvtlygml
ntlknrnpnl ktllsvggwn fgsqrfskia sntqsrrtfi
ksvppflrth gfdgldlawl ypgrrdkqhf ttlikemkae
fikeaqpgkk qlllsaalsa gkvtidssyd iakisqhldf
isimtydfhg awrgttghhs plfrgqedas pdrfsntdya
vgymlrlgap asklvmgipt fgrsftlass etgvgapisg
pgipgrftke agtlayyeic dflrgatvhr ilgqqvpyat
kgnqwvgydd qesvkskvqy lkdrqlagam vwaldld
dfqgsfcgqdlrf pltnaikdal aat
(SEQ ID NO: 1; NCBI Reference Sequence No.
NP_001267.2; 383 amino acids)

YKL-40 proteins further include the isoforms of the YKL-40 protein (SEQ ID NOs: 2-8) or a protein encoded by the mRNA of human chitinase-3-like which corresponds to SEQ ID NO: 9 described in the table below.

| | | SEQ ID |
|---|---|---|
| Sequence | NCBI Ref. No. | NO. |
| human chitinase-3-like protein 1 isoform X1 | XP_047298796.1 | 2 |
| human chitinase-3-like protein 1 isoform X2 | XP_047298797.1 | 3 |
| human chitinase-3-like protein 1 isoform X3 | XP_047298802.1 | 4 |
| human chitinase-3-like protein 1 isoform X4 | XP_047298803.1 | 5 |
| human chitinase-3-like protein 1 isoform X5 | XP_047298804.1 | 6 |
| human chitinase-3-like protein 1 isoform X6 | XP_047298829.1 | 7 |
| human chitinase-3-like protein 1 isoform X7 | XP_047298835.1 | 8 |
| human chitinase-3-like nucleic acid | NM_001276.4 | 9 |

In some embodiments, an antisense molecule can be used to neutralize YKL-40. As used herein, an “antisense” molecule includes an RNA molecule which, by binding to a complementary sequence in RNA, inhibits the function and/or completion of synthesis of the latter molecule. In some embodiments, the antisense molecule can target a nucleic acid that encodes one of SEQ ID NOs: 1-8 or a fragment thereof (e.g., amino acids 22-240 of SEQ ID NO: 1 or a fragment thereof). In some embodiments the antisense molecule can target a mRNA sequence corresponding to SEQ ID NO: 9 or a fragment thereof.

Additional YKL-40 blockade agents include small molecule inhibitors of YKL-40. Suitable small molecule inhibitors of YKL-40 are known in the art and include, but not limited to, K284 (also known as K284-6111, i.e., 2-({3-[2-(1-cyclohexen-1-yl)ethyl]-6,7-dimethoxy-4-oxo-3,4-dihydro-2-quinazolinyl}-sulfanyl)-N-(4-ethylphenyl)butanamide) (Kang et al., 2020; Jeon et al., 2021); and G721 (also known as G721-0282, i.e., N-allyl-2-[-6-butyl-1,3-dimethyl-2,4-dioxo-1,2,3,4-tetrahydropyrido[2,3-D]pyrimidin-5-yl)sulfanyl]-acetamide. Both K284 and G721 are commercially available from ChemDiv, Inc. (San Diego, California, United States of America).

By “MCP-1” it is meant the 76 amino acid sequence referenced in NCBI record accession No. NP_002973 and variously known as MCP (monocyte chemotactic protein), chemokine (C-C motif) ligand 2 (CCL2), SMC-CF (smooth muscle cell chemotactic factor), LDCF (lymphocyte-derived chemotactic factor), GDCF (glioma-derived monocyte chemotactic factor), TDCF (tumor-derived chemotactic factors), HCI1 (human cytokine 11), and MCAF (monocyte chemotactic and activating factor). A variety of suitable MCP-1 blockade agents are known in the art. These include anti-MCP-1 antibodies (e.g., anti-MCP-1 mAB) and fragments thereof as well as antisense compounds. MCP-1 blockade agents further include MCP-1 antagonist small molecules that can inhibit MCP-1 activity. Such compounds are known in the art, such as indole derivatives, cyclic amine derivatives, ureido derivatives, heterocyclics, anilides, and functional pyrroles with the ability to block MCP-1 binding to a receptor (CCR2B), and/or inhibition of MCP-1 itself, as disclosed in PCT publications WO 9905279 (1999), WO 9916876 (1999), WO 9912968, WO 9934818, WO 9909178, WO 9907351, WO 9907678, WO 9940913, WO 9940914, WO 0046195, WO 0046196, WO 0046197, WO 0046198, WO 0046199, WO 9925686, WO 0069815, WO 0069432, WO 9932468, WO 9806703, WO 9904770, WO 99045791, each of which is entirely incorporated herein by reference in its entirety. For example, suitable small molecule inhibitors of MCP-1 include INCB3344 (i.e., N-[2-[[(3S,4S)-1-[4-(1,3-benzodioxol-5-yl)-4-hydroxycyclohexyl]-4-ethoxypyrrolidin-3-yl]amino]-2-oxoethyl]-3-(trifluoromethyl)benzamide (which is commercially available from MedChemExpress, LLC, Monmouth Junction, New Jersey, United States of America).

Anti-MCP-1 antibodies are known in the art and/or can be prepared using methods known in the art, for instance, using MCP-1 or a fragment thereof (or a protein encoded by the mRNA for MCP-1 or a fragment thereof), in an antigen. The amino acid sequence of human MCP-1 is:
mkvsaallcl lliaatfipq glaqpdaina pvtccynftn rkisvqrlas yrritsskcp keavifktiv akeicadpkq kwvqdsmdhl dkqtqtpkt (SEQ ID NO: 10; NCBI Reference Sequence No. NP_002973.1; 99 amino acids)
   The nucleotide sequence corresponding to MCP-1 mRNA is:

gagaggctga gactaaccca gaaacatcca attctcaaac
tgaagctcgc actctcgcct ccagcatgaa agtctctgcc
gcccttctgt gcctgctgct catagcagcc accttcattc
cccaagggct cgctcagcca gatgcaatca atgccccagt
cacctgctgt tataacttca ccaataggaa gatctcagtg
cagaggctcg cgagctatag aagaatcacc agcagcaagt
gtcccaaaga agctgtgatc ttcaagacca ttgtggccaa
ggagatctgt gctgacccca agcagaagtg ggttcaggat
tccatggacc acctggacaa gcaaacccaa actccgaaga
cttgaacact cactccacaa cccaagaatc tgcagctaac
ttattttccc ctagctttcc ccagacaccc tgttttattt
tattataatg aattttgttt gttgatgtga aacattatgc
cttaagtaat gttaattctt atttaagtta ttgatgtttt
aagtttatct ttcatggtac tagtgttttt tagatacaga
gacttgggga aattgctttt cctcttgaac cacagttcta
cccctgggat gttttgaggg tctttgcaag aatcattaat
acaaagaatt ttttttaaca ttccaatgca ttgctaaaat
attattgtgg aaatgaatat tttgtaacta ttacaccaaa
taaatatatt tttgtacaaa a
(SEQ ID NO: 11; NCBI Reference Sequence No.
NM_002982.4)

Anti-MCP-1 antibodies are also commercially available. For example, anti-MCP-1 mAb clone 5D3-F7 is available from BioLegend (San Diego, California, United States of America).

In some embodiments, an antisense molecule can be used to neutralize MCP-1. In some embodiments, the antisense molecule can target a nucleic acid that encodes SEQ ID NO: 10 or a fragment thereof (e.g., amino acids 24-99 of SEQ ID NO: 10 or a fragment thereof). In some embodiments the antisense molecule can target a mRNA sequence corresponding to SEQ ID NO: 11 or a fragment thereof.

Hyaluronan blockade agents for use in the presently disclosed methods include agents that neutralizes a hyaluronan receptor (e.g., CD44, layilin, LYVE-1 and/or RHAMM); agents that neutralizes a hyaluronan synthase (HAS); and hyaluronidases. For example, in some embodiments, the hyaluronan blockage agent comprises at least one agent that neutralizes a hyaluronan receptor. In some embodiments, the hyaluronan receptor is selected from hyaluronic acid binding protein (HABP), CD44, layilin, LYVE-1, and RHAMM. The agents can include antibodies (or fragments thereof) that bind to the hyaluronan receptor and block its binding to HA, antisense molecules (e.g., siRNA) that targets a nucleic acid that encodes a hyaluronan receptor, and soluble fragments of the hyaluronan receptors (i.e., fragments that have been cleaved from a cell surface or recombinant or synthetic (i.e., chemically synthesized) versions thereof) that can interfere with HA binding to receptors present on a cell surface. The agent can also be hyaluronan hexasaccharide (HA6).

In some embodiments, the composition comprises a therapeutically effective amount of an agent that neutralizes CD44. In some embodiments, the agent that neutralizes CD44 is an antibody (e.g., a mAb) or an antisense molecule (e.g., a siRNA). In particular, the anti-CD44 antibody is an antibody that can interfere with the binding of hyaluronan (HA) and CD44, i.e., an antibody directed against the CD44 HA binding domain. Additional agents that can neutralize CD44 by blocking CD44-HA interactions include soluble CD44 (Misra et al., 2015; Aherns et al., 2001) and HA6 (Teriete et al., 2004; Lesley et al., 2000).

Anti-CD44 antibodies are known in the art and/or can be prepared using methods known in the art, for instance, using a CD44 amino acid sequence or a fragment thereof in an antigen. CD44 has a number of isoforms due to alternative splicing of exons. Amino acid sequences of human CD44 (Uniprot No. P16070) include, but are not limited to, CD44 antigen isoform 1 precursor (NCBI Reference Sequence NP_000601.3; SEQ ID NO:12), CD44 antigen isoform 2 precursor (NCBI Reference Sequence NP_001001389.1; SEQ ID NO: 13), CD44 antigen isoform 3 precursor (NCBI Reference Sequence NP_001001390.1, SEQ ID NO:14), CD44 antigen isoform 4 precursor (NCBI Reference Sequence NP_001001391.1, SEQ ID NO:15); and CD44 antigen isoform 5 precursor (NCBI Reference Sequence NP_001001392.1, SEQ ID NO: 16). CD44 antigen isoform 1 precursor (also known as CD44S, SEQ ID NO: 12) is the “standard” form, without variant exons and is found on most leukocytes. Amino acids 1-20 of SEQ ID NO: 12 correspond to a signal peptide, while amino acids 21-268 of SEQ ID NO: 12 corresponds to an extracellular domain that includes the HA binding region, which corresponds to amino acids 21-178 of SEQ ID NO: 12. CD44 antigen isoform 5 precursor (also known as CD44RC, SEQ ID NO: 16) is a soluble isoform of CD44. Antisense molecules that neutralize CD44 can include siRNAs that target a nucleic acid encoding one of SEQ ID NOs: 12-16, or a fragment thereof, or that target a CD44 mRNA sequence or fragment thereof, such as one of the nucleic acid sequences corresponding to NCBI Reference Nos: NM_000610.4 (SEQ ID NO: 17), NM_001001389.2 (SEQ ID NO: 18), NM_001001390.2 (SEQ ID NO: 19), NM 001001391.2 (SEQ ID NO: 20), and NM_001001392.2 (SEQ ID NO:21) or a fragment thereof. Methods of preparing agents that neutralize CD44 are also described e.g., in U.S. Patent Application Publication Nos. 2010/0092484, 2015/0044232, 2015/0374848 and 2019/0107538; the disclosure of each of which is incorporated herein by reference in its entirety.

The ability of an antibody to block binding of HA and CD44 can be determined by methods that are known in the art, e.g., ability to disrupt HA-CD44 binding in a HA-CD44 binding assay or to disrupt cell binding to a HA-coated plate (Birzele et al., 2015). Exemplary antibodies that are known to block the HA-binding domain of human CD44 include, for example, 5F12 (Teriete et al., 2004), RG7356 (Birzele et al., 2015), 15C6 and BU-75 (Sugiyama et al., 1999). In some embodiments, the anti-CD44 antibody is selected from RG7356 (commercially available, for example as catalog number TAB-128CL, Creative Biolabs, Shirley, New York, United States of America), MA4400 (also known as Hermes-1, commercially available, for example, as catalog number MA4400, Thermo Fisher Scientific, Waltham, Massachusetts, United States of America), IM7 (commercially available, for example, as catalog number 553131 from BD Biosciences, Franklin Lakes, New Jersey, United States of America), 5F12 (commercially available as catalog number LS-C87849 from Lifespan Biosciences, Seattle, Washington, United States of America, and as catalog number MA5-12394 from Thermo Fisher Scientific, Waltham, Massachusetts, United States of America), 15C6 (commercially available, for example, as catalog number LS-C179402 from Lifespan Biosciences, Seattle, Washington, United States of America), and BU-75 (commercially available, for example, as catalog number 153222 from Ximbio, London, United Kingdom).

Soluble CD44 for use according to the presently disclosed methods include recombinant proteins comprising or consisting of the extracellular domain of CD44, as well as fragments and/or Fc-fusion proteins thereof. Recombinant human CD44 is commercially available, including as a Fc fusion protein (e.g., as catalog number 3660-CD, Bio-Techne, Minneapolis, Minnesota, United States of America). In some embodiments, the soluble CD44 that neutralizes CD44 is a recombinant protein comprising or consisting of the HA binding domain of CD44 (i.e., amino acids 20-178 of SEQ ID NO: 12, Teriete, 2004) or portions thereof, e.g., amino acids 20-169 of SEQ ID NO: 12. Another entity that can interfere with HA-CD44 binding is HA6, which is commercially available, for example, from Cosmo Bio USA, Inc. (Carlsbad, California, United States of America; catalog number CSR-11007). HA6, for example, is a hexasaccharide that comprises three repeats of the disaccharide of D-glucuronic acid and N-acetyl-D-glucosamine.

In some embodiments, the blockade agent is an antibody that binds to HABP, such as the anti-HABP antibody available from Creative Diagnostics (CABT-L4621, clone CYE112, Creative Diagnostics, Shirley, New York, United States of America).

As noted hereinabove, blockade agents that neutralize a hyaluronan receptor also include agents that neutralize layilin (also known as LAYN) (Bono et al., 2001), LYVE-1 (Lawrance et al., 2016), and RHAMM (Zaman et al., 2005). Thus, agents that neutralize a hyaluronan receptor also include antibodies to layilin, LYVE-1, or RHAMM (i.e., antibodies that block HA interactions with these receptors) and fragments thereof; as well as soluble layilin, LYVE-1, or RHAMM proteins that can block HA binding to HA receptors on cell surfaces, antisense molecules (siRNAs) for nucleic acids encoding these receptors, and HA6. Amino acid sequences of human layilin (Uniprot No. Q6UX15) include the amino acid sequence of layilin isoform 1 (the longest form of layilin, NCBI Reference Sequence NP_001245319.1; SEQ ID NO: 22) and of layilin isoform 2 (NCBI Reference Sequence No. NP_849156.1, SEQ ID NO: 23). Recombinant human layilin protein can be used to neutralize HA receptors and is commercially available, for example, as catalog number 10208-H08H from Sino Biological US, Inc. (Wayne, Pennsylvania, United States of America). Human LYVE-1 (Uniprot No. Q9Y5Y7.2) has a 322 amino acid sequence (NCBI Reference Sequence NP_006682.2, SEQ ID NO:24), with a HA binding domain at amino acids 34-174 of SEQ ID NO:24. Commercially available antibodies that block HA interaction with LYVE-1 include catalog numbers MAB 20891 and MAB 20893 from Bio-Techne Corporation (Minneapolis, Minnesota, United States of America). See also, Lawrance et al., 2016. Recombinant human LYVE-1 is commercially available, for example, as catalog number 2089-LY from Bio-Techne Corporation (Minneapolis, Minnesota, United States of America). Human RHAMM (Uniprot No. 075330) is also known as cluster of differentiation 168 (CD168) and hyaluronan-mediated motility receptor (HMMR) has multiple isoforms, including isoform b (NCBI Reference Sequence NP_036616.2; SEQ ID NO: 25) with 724 amino acids. Recombinant human RHAMM is commercially available, for example, as catalog number TP313373 from Origene (Rockville, Maryland, United States of America), corresponding to the amino acid sequence for RHAMM isoform c (NCBI Reference Sequence NP_036617.2; SEQ ID NO: 26). Anti-RHAMM antibodies have also been described (Zaman et al., 2005). Antisense molecules that neutralize layilin, LYVE-1, or RHAMM include siRNAs that target a nucleic acid encoding one of SEQ ID NOs: 22-26, or a fragment thereof, or that target a layilin, LYVE-1, or RHAMM mRNA sequence or fragment thereof.

In some embodiments, the hyaluronan blockade agent includes at least one agent that neutralizes HAS. In some embodiments, the HAS is hyaluronan synthase 1 (HAS1). In some embodiments, the agent is an antibody (e.g., a mAb), an siRNA, or a small molecule inhibitor (e.g., 4-methylumbelliferone). Anti-HAS antibodies are known in the art and/or can be prepared using methods known in the art, for instance, using a HAS amino acid sequence or a fragment thereof in an antigen. In some embodiments, the agent is an antibody that targets one of the amino acid sequences of human HAS1 (Uniprot No. Q92839) (e.g., an amino acid sequence corresponding to NCBI Reference Sequence. NP_001284365.1 (SEQ ID NO:27) or NP_001514.2 (SEQ ID NO: 28) or a fragment thereof), human hyaluronan synthase 2 (HAS2; Uniprot No. Q92819) (e.g., an amino acid sequence corresponding to NCBI Reference Sequence NP_005319.1 (SEQ ID NO: 29) or a fragment thereof), or an amino acid sequence of human hyaluronan synthase 3 (HAS3; Uniprot No. 000219) (e.g., an amino acid sequence corresponding to NCBI Reference Sequence NP_001186209.1 (SEQ ID NO: 30) or NP_619515.1 (SEQ ID NO: 31), or a fragment thereof). SiRNAs for HAS can be siRNAs that target a nucleic acid encoding one of SEQ ID NOs: 27-31, or a fragment thereof, or that target a HAS mRNA sequence corresponding to one of the sequences of NCBI Reference Sequences: NM_001297436.2 (SEQ ID NO: 32), NM_001523.4 (SEQ ID NO: 33), NM_005328.3 (SEQ ID NO: 34), NM_001199280.2 (SEQ ID NO: 35), NM_005329.3 (SEQ ID NO: 36) and NM_138612.3 (SEQ ID NO:37) or a fragment thereof. Methods of preparing agents that neutralize HAS are also described e.g., in U.S. Patent Application Publication No. 2012/0009193, the disclosure of which is incorporated by reference in its entirety.

In some embodiments, the hyaluronan blockage agent comprises or further comprises a hyaluronidase. Hyaluronidase hydrolyses hyaluronic acid. According to their enzymatic mechanism, hyaluronidases are hyaluronoglucosidases (EC 3.2.1.35), i.e., they cleave the (β1-4)-linkages between N-acetylglucosamine and glucuronate. The term “hyaluronidase” can also refer to hyaluronoglucuronidases (EC 3.2.1.36), which cleave (β1-3)-linkages. Hyaluronidases are known in the art for use in ophthalmic surgery and for use in increasing absorption of parenteral fluids. Hyaluronidase can be commercially obtained from animals, where it is typically extracted from ovine or bovine testicles, leech, or bacteria. Commercially available hyaluronidases include the ovine hyaluronidase sold under the trade name VITRASE® (Bausch & Lomb, Rochester, New York, United States of America) and the bovine hyaluronidases sold under the trade names AMPHADASE® (Amphastar Pharmaceuticals Inc., Rancho Cucamonga, California, United States of America) and HYDRASE® (Akorn Inc., Lake Forest, Illinois, United States of America). Hyaluronidase can also be obtained recombinantly, e.g., by genetically manipulating human recombinant DNA in Chinese hamster ovary cells. Commercially available recombinant hyaluronidase is available under the tradenames HYLENEX® and ENHANZE™ (Halozyme Therapeutics, Inc., San Diego, California, United States of America).

In some embodiments, the presently disclosed method comprises administering (i), (ii), and (iii); i.e., at least one agent selected from the group comprising, consisting of, or consisting essentially of a steroid (e.g., dexamethasone or prednisone), an antiviral agent (e.g., remdesivir), baricitinib, an IL-6 inhibitor, and an anti-SARS-CoV-2 mAb; at least one of dupilumab or a Fab, F(ab′)₂, or scFv thereof, and at least one of the group comprising YKL-40 blockade agent, a MCP-1 blockade agent, and a hyaluronan blockade agent. In some embodiments, the method comprises administering remdesivir, dupilumab, and a YKL-40 blockade agent. In some embodiments, the method comprises administering remdesivir, dupilumab, and a MCP-1 blockade agent.

In some embodiments, the human subject in need of treatment according to the presently disclosed methods is a human subject who has tested positive for a SARS-CoV-2 infection within the last 14 days and/or is hospitalized for the treatment of COVID-19. In some embodiments, the human subject is a human subject with a positive RT-PCR for SARS-CoV-2 within the last 14 days. In some embodiments, the human subject is a human subject with a positive RT-PCR for SARS-CoV-2 within the last 14 days who is also hospitalized for treatment of COVID-19. In some embodiments, the human subject is a human subject with evidence of moderate to severe COVID-19 infections as defined by the NIH COVID-19 Severity Categorization.

In some embodiments, the subject is a subject who has been diagnosed with COVID-19 or a SARS-CoV-2 infection and is at increased risk for severe COVID-19 (e.g., risk for disease requiring hospitalization, ventilation or oxygen support, and/or that results in death). Subjects with increased risk of severe disease include human subjects over the age of 45 or over the age of 65. Underlying medical conditions or comorbidities that can result in higher risk of severe COVID-19 include, but are not limited to, cancer, chronic kidney disease, chronic lung disease (e.g., chronic obstructive pulmonary disease (COPD), asthma, cystic fibrosis, pulmonary hypertension, and interstitial lung disease), dementia, diabetes, Down's syndrome, heart disease (e.g., heart failure, coronary artery disease, hypertension, etc.), HIV, a compromised or weakened immune system, liver disease (e.g., cirrhosis), being overweight (i.e., having a body mass index (BMI) >25 kg/m²) or obese (i.e., having a BMI ≥30 kg/m²), pregnancy, sickle cell disease or thalassemia, a history of smoking, stroke, and having a history of substance abuse.

In some embodiments, the human subject is a human subject who shows evidence of lower respiratory disease during clinical assessment or imaging. For example, in some embodiments, the human subject has one or more symptom of the group comprising a saturation of oxygen (SpO₂) less than about 94% on room air at sea level, lung infiltrates of greater than about 50%, a respiratory frequency of more than about 30 breaths per minute, and a ratio of arterial partial pressure of oxygen to fraction of inspired oxygen (PaO₂/FiO₂) of less than about 300 millimeters of mercury (mmHg).

In some embodiments, the human subject is a human subject with lymphopenia (i.e., a low number of white blood cells, such as B lymphocytes, T lymphocytes, and/or natural killer cells). Symptoms of lymphopenia include, for example, swollen lymph nodes, an enlarged spleen, and/or unusual infections and/or infections of longer than normal duration. The normal range of lymphocytes for adult human subjects is 1000 to 4800 per microliter of blood (i.e., 1K-4.8K/μl). In children, the normal range for lymphocytes can be higher (e.g., about 2K/μl for a 6-year-old). In some embodiments, the human subject with lymphopenia is an adult subject with a total lymphocyte count of less than about 1.5 K/μl. In some embodiments, the human subject with lymphopenia is an adult subject with a total lymphocyte count of less than about 1 K/μl. In some embodiments, total lymphocyte counts in children diagnosed with lymphopenia can be higher than about 1 K/μl or about 1.5 K/μl, but typically less than about 2 K/μl.

The presently disclosed method can improve longer term outcomes (e.g., outcomes beyond the first 28 days of treatment or the first month of treatment). In some embodiments, the administering of the presently disclosed method reduces the risk that the human subject is admitted to an intensive care unit (ICU) (e.g., for the treatment or continued treatment of COVID-19). In some embodiments, the risk is reduced compared to the risk for a human subject with COVID-19 who is only administered (i). In some embodiments, the administering reduces the risk that the human subject is admitted to an ICU for the treatment or continued treatment of COVID-19 within a time period of about 60 days or more following the administering. Thus, for example, the method can reduce risk that the subject is admitted to the ICU for COVID-19 related treatment over the course of the first two, three, four, five, six, seven, eight, nine, ten, eleven, or twelve months after the initial administration of (i)-(iii). In some embodiments, the method comprises administering (ii) and either or both of (i) and (iii) and the method can reduce risk that the subject is admitted to the ICU for COVID-19 related treatment over the course of the first two, three, four, five, six, seven, eight, nine, ten, eleven or twelve months after the initial administration of (ii). In some embodiments, the reduced risk is for the time period including the first 60 days following an initial administration of (ii). In some embodiments, the reduced risk is for the time period including the first 360 days following an initial administration of (ii).

In some embodiments, the method the administering reduces the risk of death, optionally wherein the administering reduces the risk of death due to COVID-19, e.g., compared to a human subject who is only administered (i). In some embodiments, the reduced risk of death is for a time period of about 60 days or more following the administering, e.g., the initial administration of (ii). In some embodiments, the period of reduced risk comprises the first two, three, four, five, six, seven, eight, nine, ten, eleven or twelve months after the initial administration of (ii). In some embodiments, the reduced risk is for the time period including the first 60 days following an initial administration of (ii). In some embodiments, the reduced risk is for the time period including the first 360 days following an initial administration of (ii).

In some embodiments, the administering reduces a plasma level of YKL-40 and/or MCP-1, e.g., compared to the corresponding plasma level at the time of initial administering. In some embodiments, the administering reduces a plasma level of YKL-40 and/or MCP-1 compared to the corresponding plasma level at the time of initial administering of (ii). In some embodiments, the administering reduces hyaluronan synthesis, e.g., compared to the level of hyaluronan synthesis at the time of initial administering. In some embodiments, the administering reduces hyaluronan synthesis compared to the level at the time of initial administering of (ii).

In some embodiments, the presently disclosed subject matter provides a method of treating COVID-19 in a subject in need thereof, e.g., a human subject in need thereof, e.g., a human subject suffering from and/or diagnosed with a SARS-CoV-2 infection. In some embodiments, the method comprises administering to the subject a composition comprising a YKL-40 blockade agent, a composition comprising a MCP-1 blockade agent, a composition comprising a hyaluronan blockage agent, or any combination thereof. In some embodiments, the composition comprises a YKL-40 blockade agent and/or a MCP-1 blockade agent. Suitable blockade agents are described hereinabove. In some embodiments, the YKL-40 blockade agent and/or the MCP-1 blockade agent is an antibody or a fragment thereof. In some embodiments, the YKL-40 blockade agent and/or the MCP-1 blockage agent is a mAb. In some embodiments, the composition comprises a YKL-40 blockade agent. In some embodiments, the composition comprises an anti-YKL-40 mAb.

In some embodiments, the method further comprises administering at least one standard of care agent for COVID-19 treatment, such as at least one agent of (i), above. In some embodiments, the method comprises administering a YKL-40 blockade agent and a standard of care agent (e.g., a steroid, remdesivir or another antiviral agent, an anti-SARS-CoV-2 antibody, etc.). In some embodiments, the method comprises administering a MCP-1 blockade agent and a standard of care agent.

In some embodiments, the presently disclosed subject matter provides a method of treating COVID-19 in a human subject in need thereof, wherein said human subject is a subject diagnosed with COVID-19 and who has lymphopenia, wherein the method comprises administering at least two of: (i) one or more of the group comprising, consisting essentially of, or consisting of a steroid (e.g., dexamethasone or prednisone), an antiviral agent (e.g., remdesivir), baricitinib, an IL-6 inhibitor, and an anti-SARS-CoV-2 mAb; (ii) a composition comprising dupilumab or a Fab, F(ab′)₂, or scFv thereof; and (iii) a composition comprising a YKL-40 blockade agent, a composition comprising a MCP-1 blockade agent, a composition comprising a hyaluronan blockade agent, or any combination thereof. In some embodiments, the method comprises administering (ii) and either (i) or (iii). In some embodiments, the method comprises administering (i) and (ii). In some embodiments, the method comprises administering dupilumab and remdesivir. In some embodiments, the method further comprises administering a steroid (e.g., dexamethasone or prednisone). In some embodiments, the method comprises administering dupilumab and a steroid. In some embodiments, the method further comprises administering an YKL-40 blockade agent or a MCP-1 blockade agent. In some embodiments, the method provides enhanced survival after 30 days or after 60 days or more (e.g., after 360 days) compared to a subject diagnosed with COVID-19 and having lymphopenia who is treated with (i) only.

The compositions (e.g., antibodies) of the presently disclosed subject matter are administered to the subject in a therapeutically effective amount (i.e., an amount that has a desired therapeutic effect). Typically, antibodies can be administered parenterally. However, the compositions can be administered via any suitable route. The dose and dosage regimen can depend upon the degree of disease severity, the characteristics of the particular therapeutic agent used, e.g., its therapeutic index, the patient, and the patient's history. In some embodiments, an antibody agent of the presently disclosed methods is administered continuously over a period of 1-2 weeks.

In some embodiments, a therapeutic dose of an antibody of the presently disclosed subject matter, including, but not limited to, monoclonal antibodies, chimeric antibodies, humanized antibodies, various kinds of fragments, and biologically active homologs and fragments thereof, is from about 0.1 mg/dose to about 5,000 mg/dose or from about 0.2 mg/dose to about 1,000 mg/dose. Doses can also be administered based on body weight, for example at a dosage ranging from about 0.01 mg/kg to about 1,000 mg/kg body weight or from about 0.1 to about 500 mg/kg.

The total amount to be administered during a day can be divided into lower doses and administered at multiple times/day. In some embodiments, the method is useful for low dose treatment. In some embodiments, the method is useful for short-term treatment. For example, if 20 mg/kg/day is the prescribed amount for the day, that amount can be divided into more than one dose for administration during the day, such as doses of 10 mg/kg administered twice. In some embodiments, treatment can be as short as 1 day. In some embodiments, even doses as low as 0.01, 0.1, 0.5, 1.0, 1.5, 2.0, 2.5, 3.0, 3.5, 4.0, 4.5, and 5.0 mg/kg/day can be administered as partial doses multiple times in a day when it is determined that the entire daily dose does not need to be administered in one bolus or that it would be better to administer the daily dose in several increments.

One of ordinary skill in the art can determine the best route of administration of a pharmaceutical composition of the presently disclosed subject matter. For example, administration can be direct, enteral, or parenteral. Enteral includes, for example, oral and rectal administration. Parenteral includes, for example, intravenous administration. One of ordinary skill in the can determine the method and site of administration. For example, enteral, parental, direct, intravenous, or subcutaneous injection a composition comprising a protein agent (or biologically active fragments or homologs thereof), such as an antibody or cytokine, can be an effective treatment.

A compound or composition of the presently disclosed subject matter can be administered once or more than once. It can be administered once a day or at least twice a day. In one aspect, a compound is administered every other day within a chosen term of treatment. In one embodiment, at least two compounds of the presently disclosed subject matter are used. One of ordinary skill in the art can determine how often to administer a compound of the presently disclosed subject matter, the duration of treatment, and the dosage to be used.

As described hereinabove, the presently disclosed subject matter can, in some embodiments, relate to the administration of at least two active therapeutic agents, i.e., at least one agent selected from at least two of groups (i)-(iii) described hereinabove. When two or more agents are to be administered, they can be administered in the same pharmaceutical composition or in separate pharmaceutical compositions. When administered in separate pharmaceutical compositions, they can be administered at about the same time or at different times (different hours or days). They can be administered in any suitable order. The amount of time between administration of the different agents can vary and can be determined by one of ordinary skill in the art. For example, the two agents can be administered up to 10 minutes apart, up to 30 minutes apart, up to 1 hour apart, etc. In some embodiments, the two or more agents are administered on different days (e.g., at least one day apart). In some embodiments, one or more of the two or more agents can be administered more than once. Thus, in some embodiments, at least one agent is administered at least twice. In some embodiments, at least one agent is administered at least three, four, or five times or more. In some embodiments, duration of treatment (i.e., the time period from the initial administration of the first agent (i.e., the first dose of the first agent from groups (i)-(iii)) to the final administration of the last agent (i.e., the last dose of the last therapeutic agent from the groups (i)-(iii)) is up to about 1 week, 2 weeks, 3 weeks, 4 weeks or more.

In some embodiments, the presently disclosed subject matter provides a composition for use in the treatment of COVID-19 in a subject in need thereof, e.g., a human subject in need thereof, e.g., a human subject suffering from and/or diagnosed with a SARS-CoV-2 infection. In some embodiments, the composition comprises at least two of: (i) one or more of the group comprising, consisting essentially of, or consisting of a steroid, an antiviral agent (e.g., remdesivir), baricitinib, an interleukin-6 inhibitor, and an anti-SARS-CoV-2 monoclonal antibody; (ii) a composition comprising dupilumab or a Fab, F(ab′)2, or scFv fragment thereof; and (iii) a composition comprising a YKL-40 blockade agent, a composition comprising a MCP-1 blockade agent, a composition comprising a hyaluronan blockade agent, or any combination thereof. In some embodiments, the composition comprises (ii) (e.g., dupilimab) and either (i) or (iii). In some embodiments, the composition comprises (iii) and either (i) or (ii). In some embodiments, the composition comprises (i), (ii), and (iii). The recited components of (i), (ii), and (iii) can be provided in a single composition or separate sub-compositions. When provided in separate sub-compositions, the sub-compositions (e.g., a sub-composition comprising (i), a sub-composition comprising (ii), and/or a sub-composition comprising (iii)) can be administered at about the same time (e.g., within the same hour or within a few minutes of each other) or at different times, using the same route or different routes. The sub-compositions can be administered in any suitable order. In some embodiments, the composition comprises multiple doses of at least one of (i)-(iii), for administration at different times (e.g., on different days).

In some embodiments, the human subject is a human subject who has tested positive for a SARS-CoV-2 infection within the last 14 days and/or is hospitalized for the treatment of COVID-19. In some embodiments, the human subject shows evidence of lower respiratory disease during clinical assessment or imaging. In some embodiments, the human subject has one or more symptom of the group comprising: a SpO₂) less than about 94% on room air at sea level, lung infiltrates of greater than about 50%, a respiratory frequency of more than about 30 breaths per minute, and a ratio PaO₂/FiO₂ of less than about 300 mmHg.

In some embodiments, the composition is for use in treating a human subject having lymphopenia. In some embodiments, the composition comprises dupilumab and at least one of the group of agents recited for (i), e.g., remdesivir or another antiviral agent or a steroid (e.g., dexamethasone or prednisone).

In some embodiments, the presently disclosed subject matter provides a composition for use in the treatment of COVID-19 in a subject in need thereof, e.g., a human subject in need thereof, e.g., a human subject suffering from and/or diagnosed with a SARS-CoV-2 infection. In some embodiments, the composition comprises a YKL-40 blockade agent and/or a composition comprising a MCP-1 blockade agent. In some embodiments, the composition comprises a YKL-40 blockade agent and/or a MCP-1 blockade agent. In some embodiments, the YKL-40 blockade agent and/or the MCP-1 blockade agent is a monoclonal antibody or a fragment thereof. In some embodiments, the composition comprises a YKL-40 blockade agent. The recited components can be provided in a single composition or separate sub-compositions. When provided in separate sub-compositions, the sub-compositions (e.g., a sub-composition comprising a YKL-40 blockade agent and a sub-composition comprising a MCP-1 blockade agent) can be administered at about the same time or at different times, using the same route or different routes. The sub-compositions can be administered in any suitable order. In some embodiments, the composition comprises multiple doses of at least one of a YKL-40 blockade agent and/or a MCP-1 blockade agent, for administration at different times (e.g., on different days).

### IV. Antibodies, Peptides and Proteins

Antibodies directed against proteins, polypeptides, or peptide fragments thereof of the presently disclosed subject matter can be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants can be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, antibodies or antisera, directed against YKL-40, MCP-1, or hyaluronan, or a receptor thereof, are useful for reducing the severity of COVID-19 over a two month or longer period following diagnosis or initiation of treatment.

Fragments of YKL-40, MCP-1, hyaluronan, and the receptors thereof can be generated and antibodies prepared against the fragments. For the preparation of monoclonal antibodies, any technique which provides for the production of antibody molecules by continuous cell lines in culture can be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) can be employed to produce human monoclonal antibodies. In some embodiments, monoclonal antibodies are produced in germ-free animals.

In some embodiments, human antibodies can be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of YKL-40, MCP-1, or hyaluronan polypeptides or receptor polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

In some embodiments, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide can be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein can be cloned and sequenced using technology which is available in the art, and is described, for example, in Wright et al. (1992, Critical Rev. in Immunol. 12(3,4):125-168) and the references cited therein. Further, the antibody of the presently disclosed subject matter can be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures presented above describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA can be generated following the procedures described in Marks et al., 1991, J. Mol. Biol. 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions can be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the presently disclosed subject matter can include, but are not limited to, polyclonal, monoclonal, chimeric (i.e., “humanized”), and single chain (recombinant) antibodies, Fab fragments, and fragments produced by a Fab expression library.

The peptides of the presently disclosed subject matter can be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis can be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, can also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein can be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### Example

The following Example has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter.

### Dupilumab Trial Design:

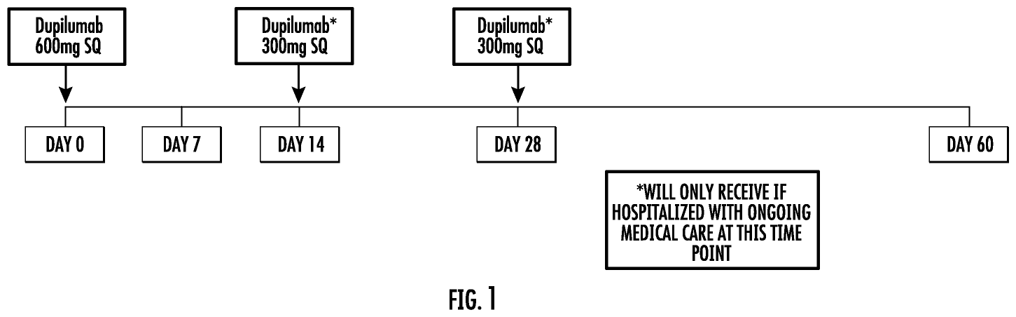

A randomized, double-blind, placebo-controlled trial was designed to assess the safety and efficacy of dupilumab use in hospitalized patients with moderate to severe COVID-19 infection. Forty eligible subjects were enrolled and randomized at a 1:1 ratio to receive either dupilumab or placebo, stratifying on disease severity measured by an oxygen requirement of ≤15 liters per minute (L/min) or >15 L/min by nasal cannula. Included were those over the age of 18 who were hospitalized with a positive reverse transcription polymerase chain reaction test (RT-PCR) for SARS-CoV-2 within the last 14 days and evidence of moderate to severe COVID-19 as defined by National Institutes of Health (NIH) COVID-19 Severity Categorization (National Institutes of Health, 2020). Patients requiring mechanical ventilation at the time of enrollment were excluded. Both arms received standard of care management per current NIH COVID-19 treatment guidelines as deemed appropriate by their primary provider (National Institutes of Health, 2020). Subjects received a loading dose of dupilumab (600 mg, given as two 300 mg subcutaneous injections) or placebo on day 0 with additional maintenance doses of 300 mg or placebo given on days 14 and 28 if the subject remained hospitalized and receiving active care (US Food and Drug Administration, 2020). See FIG. 1. Subjects were followed for 360 days, with initial outcome assessments at day 28 and 60. Evaluation of immunologic and biologic outcomes of inhibition of type 2 inflammation in COVID-19 was performed. The study group was followed to determine which subject sub-populations respond or respond best to dupilumab therapy based on biomarkers and clinical variables via a precision medicine approach.

### Outcomes

The primary outcome of the study was the proportion of patients alive and free of invasive mechanical ventilation at day 28. Safety outcomes were assessed via determination of the cumulative incidence of adverse events, including those previously reported to occur with dupilumab use (i.e., injection site reactions, eye/eyelid inflammation, conjunctivitis, herpes viral infection, eosinophilia) (US Food and Drug Administration, 2020). Additional clinical endpoints included all-cause mortality at day 28 and 60, proportion of patients alive and free of invasive mechanical ventilation at 60 days, hospital length of stay (LOS), ICU LOS, change in 8-point National Institute of Allergy and Infectious Disease (NIAID) ordinal score and change in partial pressure of oxygen (PaO₂) or oxygen saturation (SaO₂) to fraction of inspired oxygen (FiO₂) ratio. Plasma inflammatory markers, including C reactive protein (CRP), ferritin and a 47-plex cytokine panel were measured at various time points during the study. Additional type 2 inflammatory markers including TARC (CCL17), YKL40, eotaxin 3 (CCL26), arginase1 (Arg1), hyaluronan, soluble ST2 and total serum immunoglobulin E (IgE) were also measured. Ferritin, CRP and IgE levels were measured in a clinical laboratory, while other biomarkers were measured by multiplex immunoassays or ELISAs depending on the analyte. SARS-CoV-2 baseline nucleocapsid (N)-protein level was measured from day 0, 2, 5, 7 and 14 available plasma of each subject using a microbead-based immunoassay, a highly sensitive detection method described in previous studies (Shan et al., 2021). Day 0 nasopharyngeal (NP) swabs obtained for assessment of SARS-CoV-2 RNA positivity via RT-PCR underwent genomic sequencing to determine the SARS-CoV-2 lineage for samples with sufficient RNA using Artic v3 primers on either a sequencing instrument sold under the tradename MISEQ® (Illumina, Inc., San Diego, California, United States of America) or sequencing instrument sold under the tradename MinIon (Oxford Nanopore Technologies, Oxford, United Kingdom) using the and categorized according to PANGOLIN and World Health Organization (World Health Organization, 2021; Baker et al., 2021). Data on patient demographics, medical history and clinical course were collected from the electronic medical record into a secured REDCap database.

### Statistical Analysis

COVID-19 hospitalization data from the hospital from which the study patients were enrolled showed that 79.5% of COVID-19 inpatients were alive and free of mechanical ventilation at 28 days under usual care. With a pre-selected sample size of 40 patients and alpha=0.1 (one sided), a difference of 17.7% should be detectable in the proportion of subjects alive and free of mechanical ventilation at 28 days with 75% power.

Primary and secondary outcomes were analyzed under the intention-to-treat (ITT) principle. Safety outcomes were analyzed in the as treated population, including subjects who were enrolled and received at least one dose of study drug. Demographics, clinical and safety outcomes were analyzed initially with the Chi-square or Fisher's exact tests for categorical measures and two-sample t-test or Wilcoxon rank sum for continuous measures, after assessment of normality. Treatment differences in ventilator free survival proportions were analyzed via logistic regression. Mortality differences were evaluated by the log-rank test and further in the Cox regression for time to death outcome. Baseline patient characteristics and known risk factors for severe disease in COVID-19, including age, sex, body mass index (BMI), comorbidities and COVID-19 vaccination status, were adjusted in regression models if initial analyses discovered imbalance in group characteristics (Williamson et al., 2020). Differences in the biomarkers between treatment groups were analyzed exploratively by t-test or Wilcoxon rank sum testing at each time point.

Mechanical ventilation as a time varying variable was included in the Cox regression for further investigation of its influence on survivability. This provided for the accounting for the significant change in mortality risk between pre- and post-intubation when a patient was placed on mechanical ventilation. Differences in the likelihood of ICU admission between the two groups was additionally by the log-rank test. Lastly, after assessment of normality, N-protein levels were split into quartiles and analyzed by treatment group for influence on mortality via log-rank test and Cox regression. Regression models were adjusted for additional medications that were most likely to influence viral load, including monoclonal antibodies and remdesivir. Longitudinal N-protein levels over the first fourteen study days were evaluated by the treatment groups using the linear mixed effects models to account for within-subject correlations.

### Results:

### Patient and Virus Characteristics

Forty patients were enrolled. 19 (47.5%) were randomized to the dupilumab group and 21 (52.5%) to the placebo group. The groups were well matched with regard to age, BMI, race, ethnicity, comorbidities, vaccination status and days from COVID-19 symptom onset to enrollment. See Table 1, below. Of the patients randomized to the dupilumab group, 17 (89.5%) had an oxygen requirement of less than 15 L/min, while of the placebo group, 18 (85.7%) had an oxygen requirement of less than 15 L/min. Patients in the placebo arm were more likely to be male compared to the dupilumab arm (76.2% vs. 36.8%). There were no significant differences in non-study COVID-19 therapies received between the treatment groups. See Table 1. Of those NP samples available for SARS-CoV-2 sequencing, 30 of 31 (96.8%) subjects had the delta variant and one subject in the placebo group had the iota variant. See Table 2, below.

TABLE 1
Patient Characteristics.

| | Placebo | Dupilumab |
|---|---|---|
| Characteristic | (n = 21) | (n = 19) |
| Age, y, median (IQR) | 63 (55-78) | 59 (44-70) |
| Sex, male | 16 (76.2) | 7 (36.8) |
| BMI, kg/m², median (IQR) | 32.3 (26-37) | 33.6 (27-42) |
| Hispanic ethnicity | 3 (14.3) | 3 (15.8) |

Race

| | | |
|---|---|---|
| White | 14 (66.7) | 13 (68.4) |
| Black | 6 (28.6) | 4 (21.1) |
| Asian | 0 (0.0) | 1 (5.3) |
| Other | 1 (4.8) | 1 (5.3) |

Comorbidities

| | | |
|---|---|---|
| Obesity | 15 (71.4) | 14 (73.7) |
| Chronic kidney disease | 7 (33.3) | 3 (15.8) |
| Asthma | 4 (19.1) | 4 (21.1) |
| Respiratory disease (COPD, | 3 (14.3) | 2 (10.5) |
| emphysema) | | |
| Diabetes | 8 (38.1) | 7 (36.8) |
| Coronary artery disease | 6 (28.6) | 3 (15.8) |
| Cardiac valvular disease | 3 (14.3) | 2 (10.5) |
| Hypertension | 10 (47.6) | 8 (42.1) |
| Congestive heart failure | 5 (23.8) | 2 (10.5) |
| Cardiac arrythmia | 4 (19.1) | 1 (5.3) |
| Depression or psychotic disorder | 3 (14.3) | 8 (42.1) |
| Malignancy | 4 (19.1) | 3 (15.8) |
| Autoimmune disease | 2 (9.5) | 2 (10.5) |
| Organ or stem cell transplant recipient | 3 (14.3) | 1 (5.3) |
| Other immunodeficiency | 1 (4.8) | 0 (0.0) |

Smoking history

| | | |
|---|---|---|
| Never | 12 (57.1) | 15 (79.0) |
| Current | 3 (14.3) | 0 (0.0) |
| Past | 6 (28.6) | 4 (21.1) |
| Days from symptom onset to study | 8.0 (6.0-10) | 7.0 (6.0-11) |
| treatment, median (IQR) | | |

Received COVID-19 vaccine

| | | |
|---|---|---|
| Moderna | 4 (19.1) | 1 (5.3) |
| Pfizer | 5 (23.8) | 4 (21.1) |
| Johnson & Johnson | 0 (0.0) | 2 (10.5) |
| None | 12 (57.1) | 12 (63.2) |

Other COVID-19 therapeutics received

| | | |
|---|---|---|
| Steroids | 20 (95.2) | 19 (100) |
| Remdesivir | 18 (85.7) | 16 (84.2) |
| IL-6 inhibitor | 0 (0.0) | 0 (0.0) |
| Janus kinase inhibitor | 4 (19.1) | 1 (5.3) |
| Monoclonal antibodies | 3 (14.3) | 2 (10.5) |

Data are presented as No. (%) unless otherwise indicated. Abbreviations: BMI, body mass index; COPD, chronic obstructive pulmonary disease; COVID-19, coronavirus disease 2019; IL-6, interleukin 6; IQR, interquartile range.

TABLE 2
World Health Organization (WHO) Variant Designations with
Pango Lineages as Subheadings, by Treatment Group.

| | Placebo (n = 21) | Dupilumab (n = 19) |
|---|---|---|
| Delta | | |
| AY.100 | 0 | 1 |
| AY.12 | 1 | 0 |
| AY.25 | 6 | 1 |
| AY.3 | 1 | 1 |
| AY.4 | 1 | 4 |
| AY.44 | 1 | 0 |
| B.1.617.2 | 5 | 8 |
| Iota | | |
| B.1.526 | 1 | 0 |
| Unknown | 5 | 3 |

There were no significant differences in cumulative adverse events observed between the treatment groups. See Table 3, below. In the dupilumab group, five subjects developed asymptomatic eosinophilia compared to one subject in the placebo group (Fisher's exact p=0.09). There were no clinical consequences, including dermatologic, gastrointestinal, pulmonary, cardiac or neurologic, attributed to the peripheral eosinophilia seen in these subjects.

TABLE 3
Adverse Events Observed Throughout the
Treatment Period by Treatment Group.

| | No. (%) | |
|---|---|---|
| Adverse Event | Placebo (n = 21) | Dupilumab (n = 19) |
| Injection site reaction | 0 (0.0) | 0 (0.0) |
| Conjunctivitis | 2 (9.5) | 0 (0.0) |
| Bacterial pneumonia | 1 (4.8) | 2 (10.5) |
| Herpes viral infection | 0 (0.0) | 0 (0.0) |
| Eosinophilia^{a} | 1 (4.8) | 5 (26.3) |
| Hyper-eosinophilic syndrome | 0 (0.0) | 0 (0.0) |
| Other infections | 2 (9.5) | 4 (21.1) |
| Cumulative | 6 | 11 |

Other infections included Clostridioides difficile infection (1), bacteremia (2), urinary tract infection (2), and oral candidiasis (1).
^{a}Eosinophilia was defined as an absolute eosinophil count >0.6 k/μL at ≥1 measurement throughout the study period. Difference between treatment groups was not statistically significant with Fisher extract p = .09.

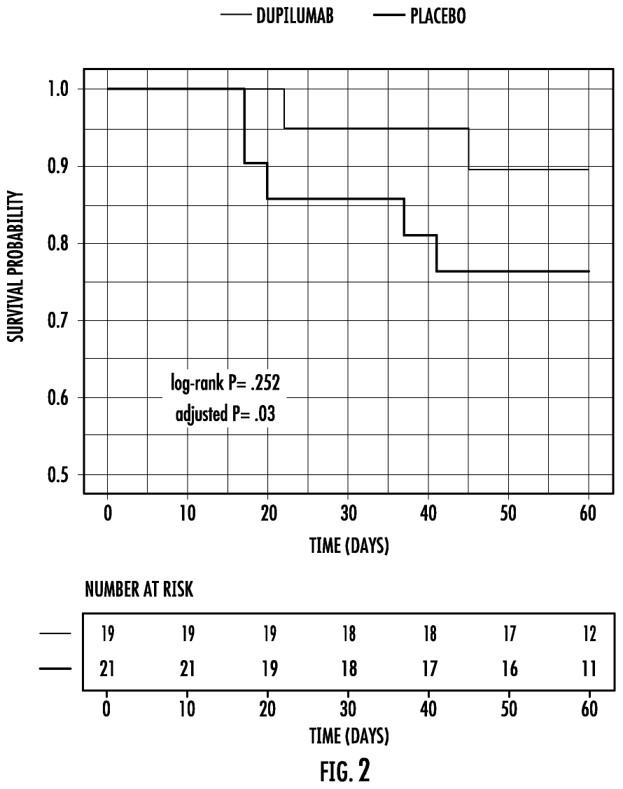

There was no significant difference in the primary endpoint of proportion of patients alive and free of mechanical ventilation at day 28 between the two groups. See Table 4, below. However, by the secondary endpoint at 60 days, 89.5% of subjects in the dupilumab group were alive compared to 76.2% for the placebo group as no patients remained on mechanical ventilation by day 60 in either group. See Table 4. After adjustment for sex and mechanical ventilation as a time varying predictor, the risk of death over 60-day follow-up period was significantly lower in dupilumab group compared to placebo (hazard ratio [THR], 0.05 [95% confidence interval {CI}, 0.004-0.72]; P=0.03. See FIG. 2 and Table 4.

TABLE 4
Primary and Secondary Endpoints, by Treatment Group.

| | Placebo | Dupilumab | OR or HR | |
|---|---|---|---|---|
| Endpoint | (n = 21) | (n = 19) | (95% CI) | P Value |
| Proportion of | 18 (85.7) | 15 (78.9) | Unadjusted | .57 |
| patients alive and | | | OR: 1.60 | |
| free of mechanical | | | (.31-8.30) | |
| ventilation by day | | | Adjusted | .34 |
| 28 | | | OR: 2.45 | |
| | | | (.40-15.10) | |
| Proportion of | 16 (76.2) | 17 (89.5) | Unadjusted | .28 |
| patients alive and | | | OR: 0.38 | |
| free of mechanical | | | (.06-2.22) | |
| ventilation by day | | | Adjusted | .40 |
| 60 | | | OR: 0.44 | |
| | | | (.07-2.96) | |
| Mortality by day | 3 (14.3) | 1 (5.3) | Unadjusted | .36 |
| 28 | | | HR: 0.35 | |
| | | | (.04-3.32) | |
| | | | Adjusted | .09 |
| | | | HR: 0.06 | |
| | | | (.003-1.59) | |
| Mortality by day | 5 (23.8) | 2 (10.5) | Unadjusted | .27 |
| 60 | | | HR: 0.40 | |
| | | | (.08-2.05) | |
| | | | Adjusted | .03 |
| | | | HR: 0.05 | |
| | | | (.004-.72) | |

Primary endpoint was ventilator-free survival by day 28. Secondary endpoints were ventilator-free survival by day 60, mortality by day 60, and mortality by day 28. Proportions are listed as No. (0%). The differences in the ventilator-free survival proportions were evaluated using logistic regression, adjusted for sex. Differences in mortality risk were evaluated in the Cox regression, adjusted for sex and time-varying mechanical ventilation. Abbreviations: CI, confidence interval; HR, hazard ratio; OR, odds ratio.

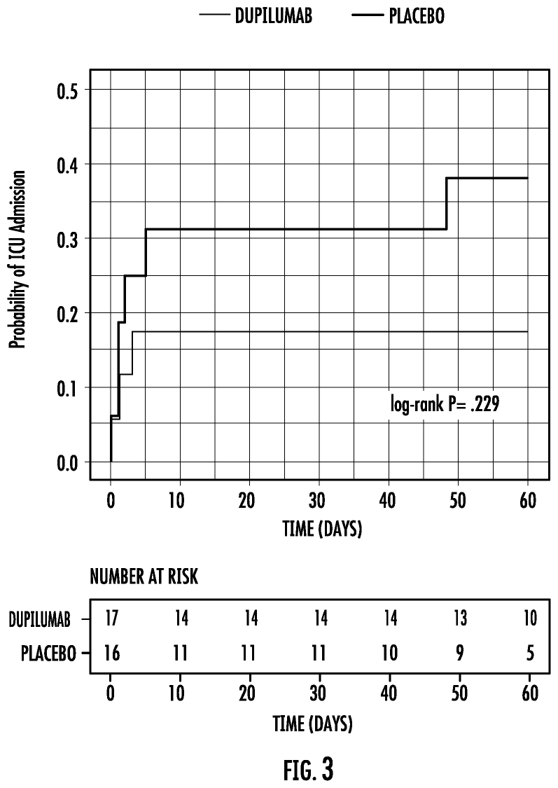

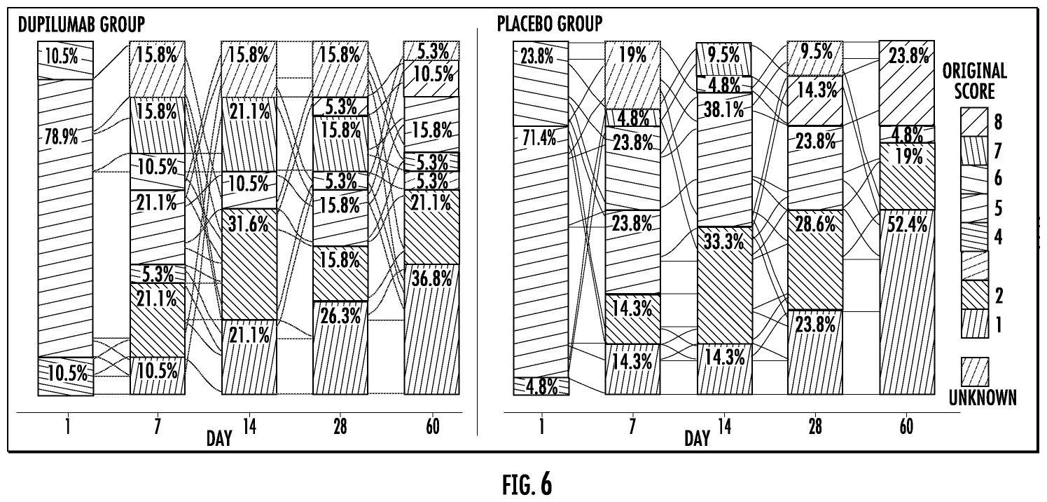

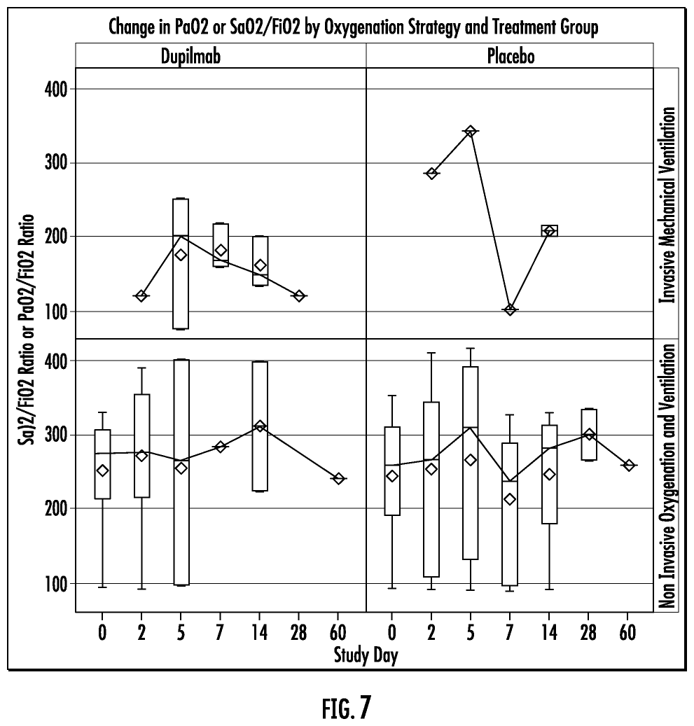

Among those not already admitted to the ICU at randomization (33 patients), numerically fewer subjects in the dupilumab group required ICU care (17.7%) compared to the placebo group (37.5%) though this difference was not statistically significant (log-rank p=0.23, HR 0.44, CI: 0.09-2.09, p=0.30 adjusted for sex). See FIG. 3. There was no difference in additional secondary endpoints between the two treatment groups. See FIGS. 6 and 7.

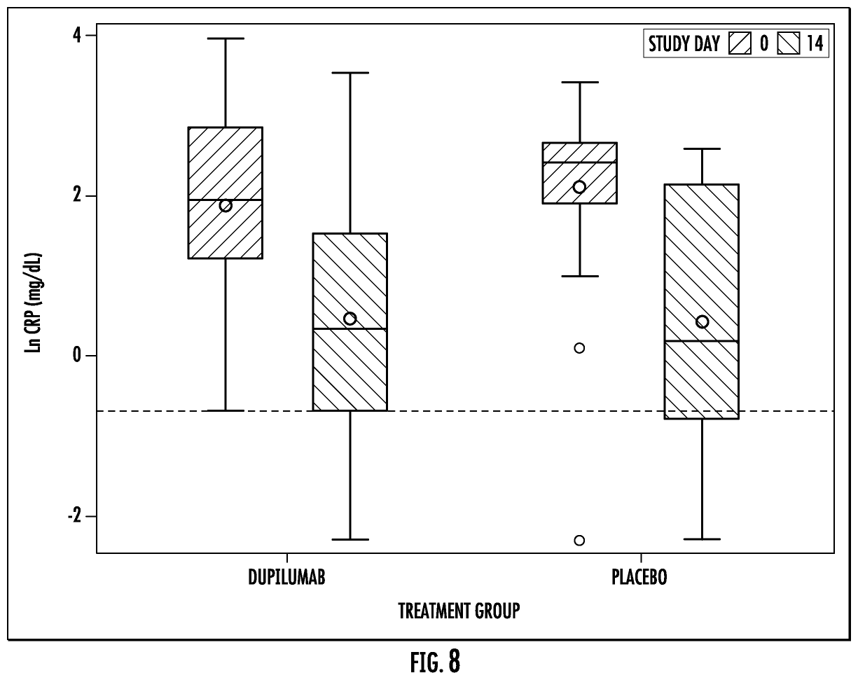

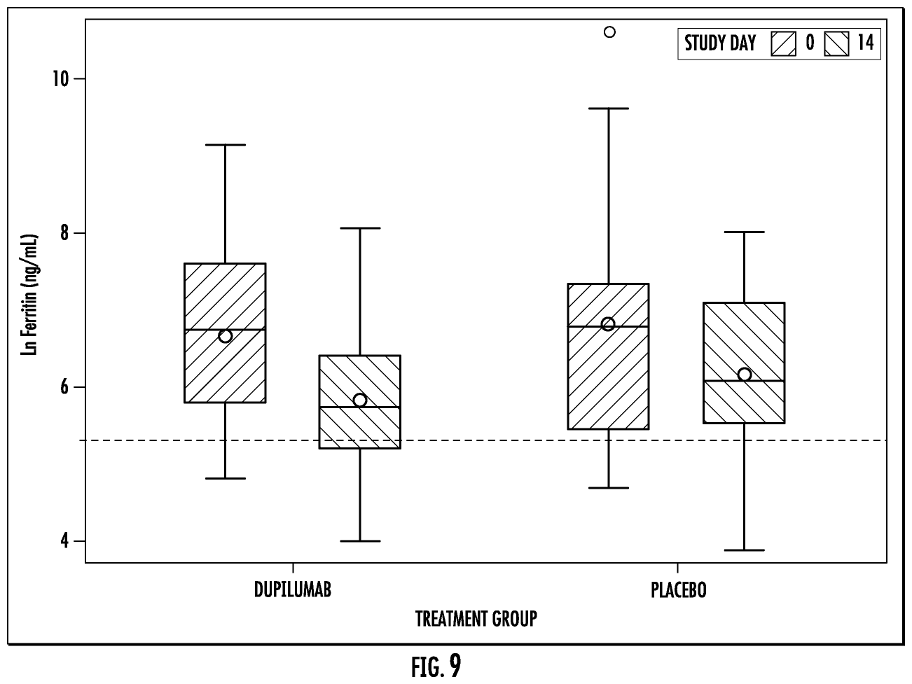

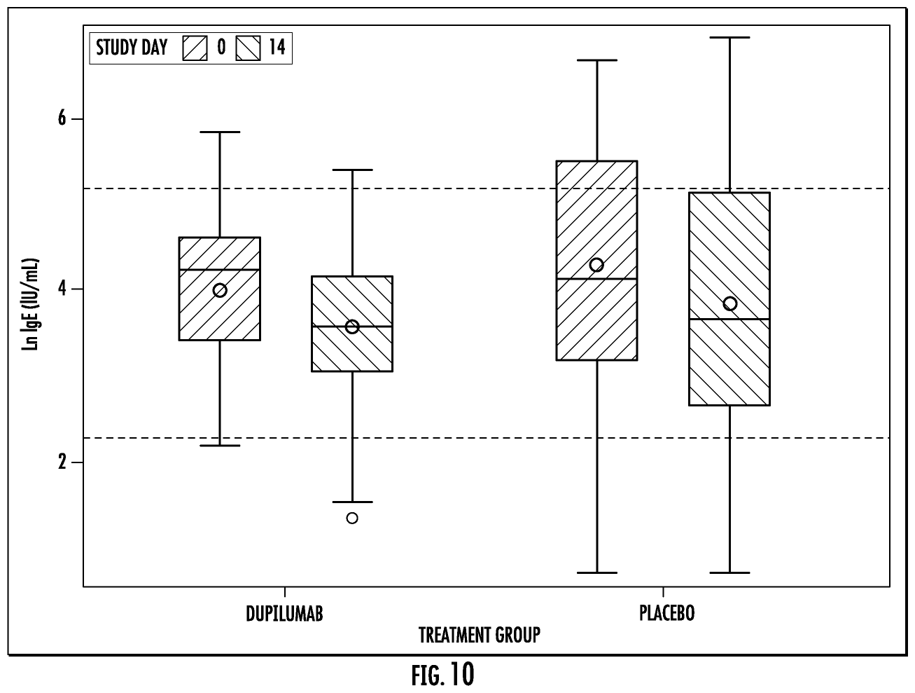

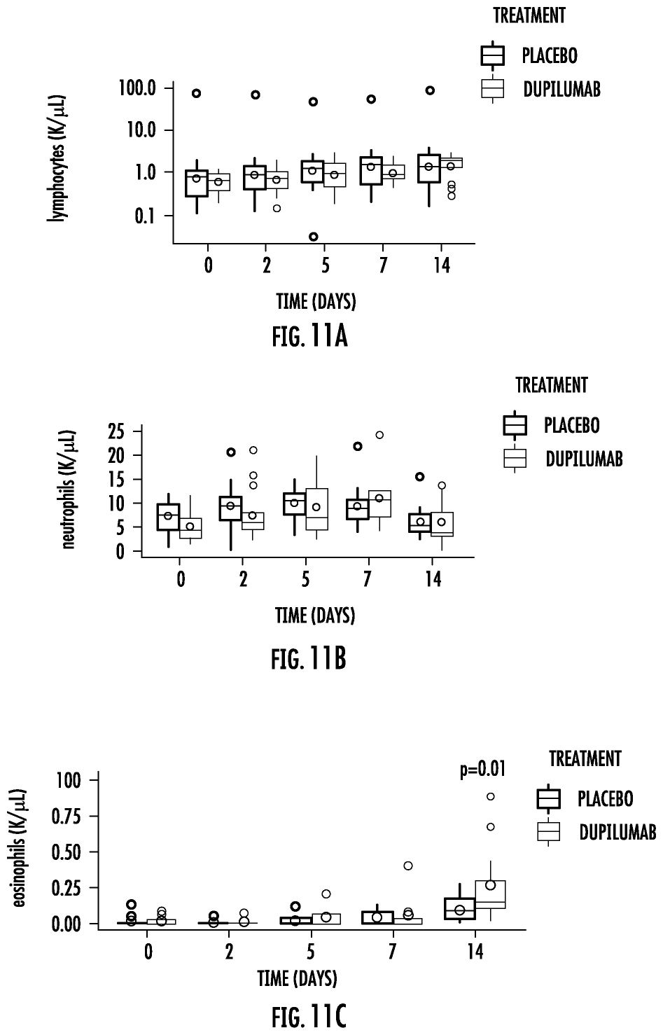

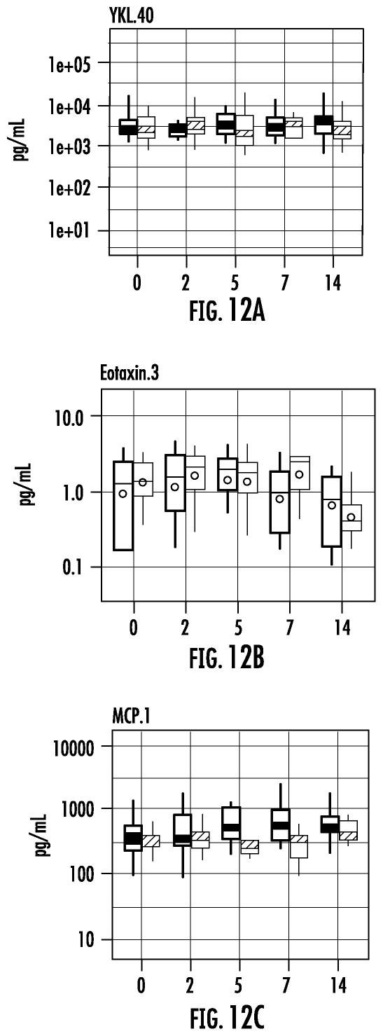

In both treatment groups, CRP, ferritin and IgE levels declined in the first two weeks with no significant difference in the change in measures from day 0 to 14 between groups. See FIGS. 8-10. When looking at the change in absolute cell counts over time (see FIGS. 11A-11C), there was an increase in eosinophils by day 14 in the dupilumab group compared to the placebo group (p=0.01 by Wilcoxon rank sum). See FIG. 11C. Analysis of patient cytokine, chemokine and growth factors in serum at various study time points showed a decreased monocyte chemoattractant protein-1 (MCP-1) at day 7 in the dupilumab treatment group compared to placebo (p=0.04 by Wilcoxon rank sum. See FIG. 12C. By day 14, there was a larger decrease in eotaxin-3 levels in the dupilumab group compared to the placebo (p=0.08 by Wilcoxon rank sum). See FIG. 12B. Additionally, there was a trend towards decreased levels of YKL40 in the dupilumab group compared to the placebo by day 14 (p=0.26 by Wilcoxon rank sum). See FIG. 12A.

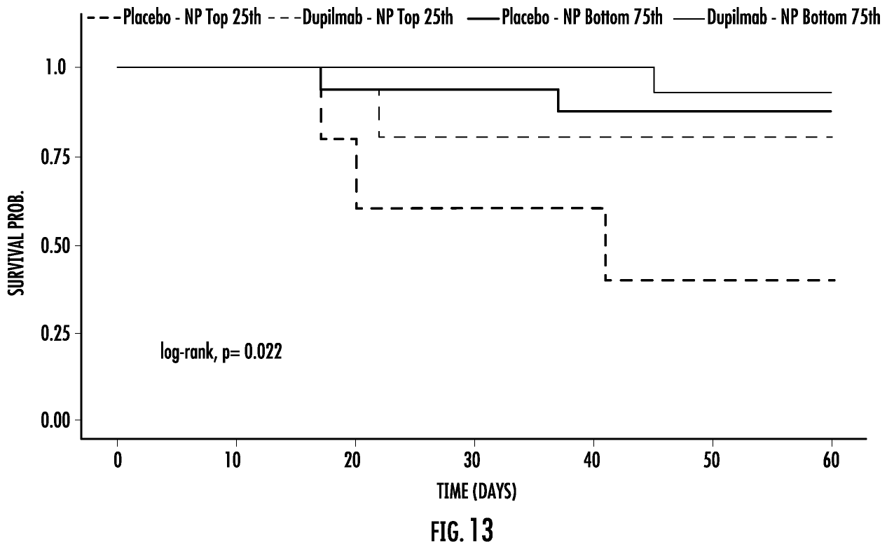

There was no statistically significant difference in baseline N-protein levels in the dupilumab group (median 671 ng/mL) compared to the placebo group (median 580 ng/mL; p=0.75 by Wilcoxon rank sum). When comparing the top quartile vs. the bottom three quartiles (i.e., bottom 75^{th} percentile) of baseline N-protein level within each treatment group, we found significant survival difference among the four groups (log-rank p=0.022). See FIG. 13. The 60-day mortality risk for those in the top quartile of baseline N-protein was 3.8 times of those in the bottom three quartiles after adjusting for treatment group, remdesivir use and monoclonal antibody use (95% CI: 0.78-18.7, p=0.098). N-protein levels in log-scale declined significantly from baseline to day 14 levels (p<0.0001), however, no difference was found in the rate of decline between the two treatment groups (p=0.17).

### Precision Medicine:

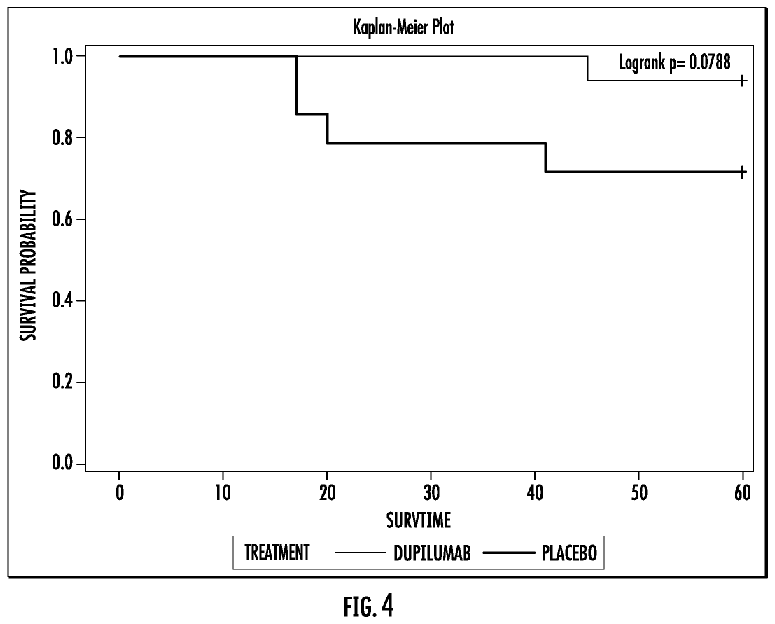

Treatment effect on mortality at 60 days was assessed via stratification by patient demographics, comorbidities, concomitant COVID-19 therapeutics received, vaccination status, days from symptom onset (dichotomized by median of 8 days) and day 0 lab work levels, including cell counts, liver function testing, renal function and inflammatory markers. Through isolation of only those patients with lymphopenia (defined as <1.0 K lymphocytes/μl blood) on study day 0 (17 patients in the dupilumab group and 14 patients in the placebo group), the association of dupilumab with decreased 60-day mortality improved (p=0.08 by log-rank, ****Cox prop adjustment for gender/vaccination status). See FIG. 4. This trend continued when the patients were followed for 360 days. As shown in FIG. 5A, 360-day mortality was improved in the dupilumab group compared to placebo based on all 40 patient but was further improved compared to placebo among the 31 patients with lymphopenia at study day 0. See FIG. 5B.

### Discussion

In this randomized double-blind placebo-controlled trial, although there was no statistical difference between study groups regarding the primary endpoint of 28-day ventilator free survival, the secondary endpoint of increased 60-day survival in the dupilumab group was achieved. Increased 360-day survival was also observed. Additionally, there were no safety signals seen with dupilumab use. The proportion of eosinophilia did trend towards the dupilumab group, which is consistent with initial dupilumab trials in asthma patients (Castro et al., 2018; Rabe et al., 2018). Importantly, there were no downstream clinical impacts observed in those patients with peripheral eosinophilia throughout the study period.

Although most deaths occurred in the placebo arm (5) compared to dupilumab (2), the overall mortality of subjects enrolled in this study (17.5%) was higher than expected, suggesting enrollment of a population with relatively higher disease severity. ICU mortality was 20% in the dupilumab group versus 36% in placebo, and ventilator mortality was 50% in the dupilumab group compared to 100% in placebo. Severity of illness seen in the present study reflected that enrollment occurred during the delta surge and that the majority of those enrolled were unvaccinated, consistent with national data at the time. For example, the National Hospital Care Survey (NHCS) data from the US Centers for Disease Control and Prevention (CDC), showed 11.9-13.1% in-hospital mortality in select hospitals throughout the United States with ventilatory mortality rates ranging from 47.9%-74.1% during the time period during which this study enrolled subjects. Furthermore, baseline N-protein levels were the same between the two groups and comparable to baseline N-protein levels of patients enrolled in the ACTIV3 trials (Lundgren et al., 2022). As high N-protein levels are predictive of COVID-19 disease progression, a finding also demonstrated in this study, this suggests patients enrolled in the study were of comparable baseline disease severity (Lundgren et al., 2022).

The longevity of moderate to critical COVID-19 was notable throughout the implementation of this trial, which is consistent with recent studies describing prolonged sequelae post-acute infection. Recent studies have reported immunologic dysfunction from COVID-19 extending out to 8 months for mild to moderate COVID-19 with deaths from severe COVID-19 occurring out to 12 months (Phetsouphanh et al., 2022; Mainous et al., 2021). These findings combined support a late clinical benefit of blockade of a type 2 immune process in COVID-19. The response to dupilumab in asthma is also protracted with improvements in FEV1 first being observed 2 weeks after initiation of treatment (Rabe et al., 2018). Thus, without being bound to any one theory, it is believed that the time to clinical effect of dupilumab in the acute COVID-19 setting can limit an ability to see early clinical differences between the treatment groups. For example, subjects in the study who ultimately required mechanical ventilation did so within the first 8 days of the study, some within 1-2 days of enrollment, during a time in which drug concentration could have been lower, particularly in the context of a rapidly evolving clinical process.

Although biomarker trends seen in both groups were likely influenced by the steroids that almost all subjects received, a reduction of the Type 2 immune markers YKL40 and eotaxin-3 was observed in the dupilumab arm when compared to the placebo arm, indicative of IL-4Rα blockade with inhibition of downstream mediators of the type 2 immune response. Increased peripheral eosinophil counts in the dupilumab group occurred by day 14, consistent with previous observations of dupilumab use in patients with atopic disease, likely due to decreased eosinophil uptake in tissue (Rabe et al., 2018; Hamilton et al., 2021). While IgE decrease was not seen at 2 weeks of dupilumab treatment, this is consistent with prior studies showing gradual decline of IgE levels compared to other biomarkers after dupilumab initiation (Hamilton, 2021). A reduction in MCP-1, a potent chemoattractant molecule of monocytes/macrophages, was also observed in the dupilumab group, high levels of which have been associated with COVID-19 disease severity (Deshmane et al., 2009; Chen et al., 2020).

The total 60-day mortality was 10.5% in the dupilumab group versus 23.8% in the placebo group. The ICU admission mortality was 20% in the dupilumab group versus 36.4% in the placebo group with 50% ventilator mortality in the dupilumab group compared to 100% in the placebo group. Overall, these numbers suggest enrollment of those with increased disease severity. The enrollment for the study occurred during a delta surge, with the majority of those enrolled unvaccinated, which was consistent with national data.

Lymphopenia has been identified in COVID-19 studies as a predictor for disease severity and need for hospitalization (Tan et al., 2020). In the present study 17/19 patients in the dupilumab group and 14/21 patients in the placebo group had low absolute lymphocyte counts on their day 0 labs. When limiting survival analysis to only those with lymphopenia, this improved the association of dupilumab with mortality reduction in the time to event mortality analysis. This suggests that dupilumab use can be more beneficial in those who present with lymphopenia, an otherwise cheap and feasible laboratory marker of disease severity in COVID-19.

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference.
Aherns et al. “Soluble CD44 inhibits melanoma tumor growth by blocking cell surface CD44 binding to hyaluronic acid.” Oncogene, 20: 3399-3408 (2001).
Aschwanden C. Five reasons why COVID herd immunity is probably impossible. Nature. 2021; 591(7851):520-522.
Birzele et al. “CD44 isoform status predicts response to treatment with anti-CD44 antibody in cancer patients.” Clinical Cancer Research, 21(12):2753-2762 (2015).
Blauvelt A, de Bruin-Weller M, Gooderham M, et al. Long-term management of moderate-to-severe atopic dermatitis with dupilumab and concomitant topical corticosteroids (LIBERTY AD CHRONOS): a 1-year, randomised, double-blinded, placebo-controlled, phase 3 trial. Lancet. 2017; 389(10086):2287-2303.
Bono et al. “Layilin, a novel integral membrane protein is a hyaluronan receptor.” Molecular Biology of the Cell, 12(4):891-900 (2001).
Castro M, Corren J, Pavord I D, et al. Dupilumab Efficacy and Safety in Moderate-to-Severe Uncontrolled Asthma. N Engl J Med. 2018; 378(26):2486-2496.
Chen Y, Wang J, Liu C, et al. IP-10 and MCP-1 as biomarkers associated with disease severity of COVID-19. Mol Med. 2020; 26(1). doi:10.1186/s10020-020-00230-x.
Deshmane S L, Kremlev S, Amini S, Sawaya B E. Monocyte Chemoattractant Protein-1 (MCP-1): An Overview. J Interf Cytokine Res. 2009; 29(6):313-326.
Donlan A N, Sutherland T E, Marie C, et al. IL-13 is a driver of COVID-19 severity. JCI Insight. 2021; 6(15). doi:10.1172/jci.insight.150107.
Faibish M, Francescone R, Bentley B, Yan W, Shao R. A YKL-40-neutralizing antibody blocks tumor angiogenesis and progression: a potential therapeutic agent in cancers. Mol Cancer Ther. 2011 May; 10(5):742-51.
Food and Drug Administration (FDA). Dupixent package insert. Published online 2020.
Jeon S H, Lee Y S, Yeo I J, Lee H P, Yoon J, Son D J, Han S B, Hong J T. Inhibition of Chitinase-3-like-1 by K284-6111 Reduces Atopic Skin Inflammation via Repressing Lactoferrin. Immune Netw. 2021 Jun. 29; 21(3):e22.
Kang, K.; Kim, K.; Lee, S.-R.; Kim, Y.; Lee, J. E.; Lee, Y. S.; Lim, J.-H.; Lim, C.-S.; Kim, Y. J.; Baek, S. I.; Song, D. H.; Hong, J. T.; Kim, D. Y. Selection and Characterization of YKL-40-Targeting Monoclonal Antibodies from Human Synthetic Fab Phage Display Libraries. Int. J. Mol. Sci. 2020, 21, 6354.
Lawrance et al. “Binding of Hyaluronan to the Native Lymphatic Vessel Endothelial Receptor LYVE-1 is Critically Dependent on Receptor Clustering and Hyaluronan Organization.” J. Biol. Chem., 291)15):8014-8030 (2016).
Lesley et al. “Hyaluronan binding by cell surface CD44.” J. Biol. Chem., 275(35):26967-26975 (2000).
Lundgren J D, Grund B, Barkauskas C E, et al. Responses to a neutralizing monoclonal antibody for hospitalized patients with COVID-19 according to baseline antibody and antigen levels. Ann Intern Med 2022; 175:234-43.
Hamilton J D, Harel S, Swanson B N, et al. Dupilumab suppresses type 2 inflammatory biomarkers across multiple atopic, allergic diseases. Clin Exp Allergy. 2021; 51(7):915-931.
Hoffmann et al. “SARS-CoV-2 cell entry depends on ACE2 and TMPRSS2 and is blocked by a clinically proven protease inhibitor.” Cell 181:271-280.e8 (2020).
Mainous A G, Rooks B J, Wu V, Orlando F A. COVID-19 Post-acute Sequelae Among Adults: 12 Month Mortality Risk. Front Med. 2021; 8. doi:10.3389/fmed.2021.778434.
Mangalmurti and Hunter. “Cytokine Storms: Understanding COVID-19.” Immunity, 53:19-25 (2020).
Misra et al. “Interactions between hyaluronan and its receptors (CD44, RHAMM) regulate the activities of inflammation and cancer.” Frontiers in Immunology, 6:201 (2015).
Mogul et al. “Baricitinib: the second FDA-approved JAK inhibitor for the treatment of rheumatoid arthritis.” Annals of Pharmacotherapy, 53(9):947-953 (2019).
NIH. Coronavirus Disease 2019 (COVID-19) Treatment Guidelines. 2020; 2019:130. covid19treatmentguidelines.nih.gov
PCT International Publication No. 98/06703.
PCT International Publication No. 99/04770.
PCT International Publication No. 99/05279.
PCT International Publication No. 99/07351.
PCT International Publication No. 99/07678.
PCT International Publication No. 99/09178.
PCT International Publication No. 99/12968.
PCT International Publication No. 99/25686.
PCT International Publication No. 99/32468.
PCT International Publication No. 99/34818.
PCT International Publication No. 99/40913.
PCT International Publication No. 99/40914.
PCT International Publication No. 00/46195.
PCT International Publication No. 00/46196.
PCT International Publication No. 00/46197.
PCT International Publication No. 00/46198.
PCT International Publication No. 00/46199.
PCT International Publication No. 00/069815.
PCT International Publication No. 00/069432.
PCT International Publication No. 2021/013884.
Pedersen & Ho. “SARS-CoV-2: a storm is raging.” J. Clin. Invest., 130: 2202-2205 (2020).
Phetsouphanh C, Darley D R, Wilson D B, et al. Immunological dysfunction persists for 8 months following initial mild-to-moderate SARS-CoV-2 infection. Nat Immunol. Published online 2022. doi:10.1038/s41590-021-01113-x.
Rabe K F, Nair P, Brusselle G, et al. Efficacy and Safety of Dupilumab in Glucocorticoid-Dependent Severe Asthma. N Engl J Med. 2018; 378(26):2475-2485.
Ritchie H, Mathieu E, Rodés-Guirao L, et al. Coronavirus Pandemic (COVID-19). OurWorldInData.org. Published 2020.
Salamon J, Hoffmann T, Elies E, Peldschus K, Johansen J S, Lüers G, et al. (2014) Antibody Directed against Human YKL-40 Increases Tumor Volume in a Human Melanoma Xenograft Model in Scid Mice. PLoS ONE 9(4): e95822.
Sugiyama et al. “CD3-Mediated T-cell activation is inhibited by anti-CD44 monoclonal antibodies directed to the hyaluronan-binding region.” Immunological Investigations, 28(2-3):185-200 (1999).
Tan L, Wang Q, Zhang D, et al. Lymphopenia predicts disease severity of COVID-19: a descriptive and predictive study. Signal Transduct Target Ther. 2020; 5(1):16-18.
Teriete et al. “Structure of the regulatory hyaluronan binding domain in the inflammatory leukocyte homing receptor CD44.” Molecular Cell, 13:483-496 (2004).
The RECOVERY Collaborative Group. “Dexamethasone in Hospitalized Patients with Covid-19—Preliminary Report.” Published online (Jul. 17, 2020) at NEJM.org doi:10.1056/NEJMoa2021436 (2020).
Thibodeaux Q, Smith M P, Ly K, Beck K, Liao W, Bhutani T. A review of dupilumab in the treatment of atopic diseases. Hum Vaccines Immunother. 2019; 15(9):2129-2139.
Tisoncik et al. “Into the eye of the cytokine storm.” Microbiology and Molecular Biology Reviews, 76(1): 16-32 (2012).
U.S. Patent Application Publication No. 2010/0092484.
U.S. Patent Application Publication No. 2012/0009193.
U.S. Patent Application Publication No. 2015/0044232.
U.S. Patent Application Publication No. 2015/0374848.
U.S. Patent Application Publication No. 2019/0107538.
U.S. Pat. No. 11,155,638.
Wenzel S, Castro M, Corren J, et al. Dupilumab efficacy and safety in adults with uncontrolled persistent asthma despite use of medium-to-high-dose inhaled corticosteroids plus a long-acting β2 agonist: a randomised double-blind placebo-controlled pivotal phase 2b dose-ranging trial. Lancet. 2016; 388(10039):31-44.
Williamson E J, Walker A J, Bhaskaran K, et al. Factors associated with COVID-19-related death using OpenSAFELY. Nature. 2020; 584(7821):430-436.
Winkler et al. “Neuropilin-1 facilitates SARS-CoV-2 cell entry and infectivity.” Science, 370:856-860 (2020).
Wynn T A. IL-13 effector functions. Annu Rev Immunol. 2003; 21:425-456.
Zaman et al. “Expression and Roles of the Hyaluronan Receptor RHAMM in Inflammation after Bleomycin Injury.” Am. J Respir. Cell Mol. Biol., 33:447-454 (2005).

It will be understood that various details of the presently disclosed subject matter may be changed without departing from the scope of the presently disclosed subject matter. Furthermore, the foregoing description is for the purpose of illustration only, and not for the purpose of limitation.

1. A method of treating coronavirus disease 2019 (COVID-19) in a human subject in need thereof, wherein the method comprises administering to the subject at least two of:
(i) one or more of a steroid, an antiviral agent, baricitinib, an interleukin-6 inhibitor, and an anti-severe acute respiratory syndrome coronavirus 2 (SARS-CoV-2) monoclonal antibody;
(ii) a composition comprising dupilumab or a Fab, F(ab′)2, or scFv fragment thereof; and
(iii) a composition comprising a chitinase-3-like protein 1 (YKL-40) blockade agent, a composition comprising a monocyte chemoattractant protein 1 (MCP-1) blockade agent, a composition comprising a hyaluronan blockade agent, or any combination thereof.

2. The method of claim 1, wherein the method comprises administering (ii) in combination with either (i) or (iii).

3. The method of claim 1, wherein the method comprises administering (iii) in combination with either (i) or (ii).

4. The method of claim 1, wherein the method comprises administering (i), (ii), and (iii).

5. The method of claim 1, wherein the human subject is a human subject who has tested positive for a SARS-CoV-2 infection within the last 14 days and/or is hospitalized for the treatment of COVID-19.

6. The method of claim 5, wherein the human subject shows evidence of lower respiratory disease during clinical assessment or imaging.

7. The method of claim 6, wherein the human subject has one or more symptom of the group consisting of a saturation of oxygen (SpO2) less than about 94% on room air at sea level, lung infiltrates of greater than about 50%, a respiratory frequency of more than about 30 breaths per minute, and a ratio of arterial partial pressure of oxygen to fraction of inspired oxygen (PaO2/FiO2) of less than about 300 millimeters of mercury (mmHg).

8. The method of claim 1, wherein the human subject is a human subject with lymphopenia.

9. The method of claim 1, wherein the administering reduces the risk that the human subject is admitted to an intensive care unit (ICU), optionally wherein the administering reduces the risk that the human subject is admitted to an ICU for the treatment of COVID-19 within a time period of about 60 days or more following the administering.

10. The method of claim 1, wherein the administering reduces the risk of death, optionally wherein the administering reduces the risk of death due to COVID-19 for a time period of about 60 days or more following the administering.

11. The method of claim 1, wherein the administering of the composition comprising dupilumab or a Fab, F(ab′)2, or scFv fragment thereof is performed subcutaneously.

12. The method of claim 11, comprising administering two or more doses of the composition that comprises dupilumab or a Fab, F(ab′)2, or scFv fragment thereof, optionally wherein said two more doses are administered about 14 days apart.

13. The method of claim 1, wherein the administering reduces a plasma level of YKL-40 and/or MCP-1.

14. The method of claim 1, wherein the administering reduces hyaluronan synthesis.

15. A method of treating coronavirus disease 2019 (COVID-19) in a human subject in need thereof, wherein the method comprises administering to the subject a composition comprising a chitinase-3-like protein 1 (YKL-40) blockade agent and/or a composition comprising a monocyte chemoattractant protein 1 (MCP-1) blockade agent.

16. The method of claim 15, wherein the YKL-40 blockade agent and/or the MCP-1 blockade agent is a monoclonal antibody.

17. Composition for use in the treatment of coronavirus disease 2019 (COVID-19) in a human subject in need thereof, wherein the composition comprises at least two of:
(i) one or more of a steroid, an antiviral agent, baricitinib, an interleukin-6 inhibitor, and an anti-severe acute respiratory syndrome coronavirus 2 (SARS-CoV-2) monoclonal antibody;
(ii) a composition comprising dupilumab or a Fab, F(ab′)2, or scFv fragment thereof; and
(iii) a composition comprising a chitinase-3-like protein 1 (YKL-40) blockade agent, a composition comprising a monocyte chemoattractant protein 1 (MCP-1) blockade agent, a composition comprising a hyaluronan blockade agent, or any combination thereof.

18. The composition for use of claim 17, wherein the composition comprises (ii) and either (i) or (iii).

19. The composition for use of claim 17, wherein the composition comprises (iii) and either (i) or (ii).

20. The composition for use of claim 17, wherein the composition comprises (i), (ii), and (iii).

21. The composition for use of claim 17, wherein the human subject is a human subject who has tested positive for a SARS-CoV-2 infection within the last 14 days and/or is hospitalized for the treatment of COVID-19.

22. The composition for use of claim 21, wherein the human subject shows evidence of lower respiratory disease during clinical assessment or imaging.

23. The composition for use of claim 22, wherein the human subject has one or more symptom of the group consisting of a saturation of oxygen (SpO2) less than about 94% on room air at sea level, lung infiltrates of greater than about 50%, a respiratory frequency of more than about 30 breaths per minute, and a ratio of arterial partial pressure of oxygen to fraction of inspired oxygen (PaO2/FiO2) of less than about 300 millimeters of mercury (mmHg).

24. The composition for use of claim 17, wherein the human subject is a human subject having lymphopenia.

25. Composition for use in the treatment of coronavirus disease 2019 (COVID-19) in a human subject in need thereof, wherein the composition comprises a chitinase-3-like protein 1 (YKL-40) blockade agent and/or a composition comprising a monocyte chemoattractant protein 1 (MCP-1) blockade agent.

26. The composition for use of claim 25, wherein the composition comprises a YKL-40 blockade agent and/or a MCP-1 blockade agent, optionally wherein the YKL-40 blockade agent and/or the MCP-1 blockade agent is a monoclonal antibody.


SEQUENCE LISTING (49 sequences)

SEQ ID NO: 1 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 2 - protein, 389 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRADFGPRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 3 - protein, 360 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRADFGPRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTICDFLRGATVHRILGQQVPYATKGNQWVGYDDQESVKSKVGFPKATPQDKEKEGGPAPSSQIPVPCTEVWAGLTEHMCPVHTLGQGTQPCSSGPPLPGAVPEGQAAGGRHGMGPGPG

SEQ ID NO: 4 - protein, 354 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTICDFLRGATVHRILGQQVPYATKGNQWVGYDDQESVKSKVGFPKATPQDKEKEGGPAPSSQIPVPCTEVWAGLTEHMCPVHTLGQGTQPCSSGPPLPGAVPEGQAAGGRHGMGPGPG

SEQ ID NO: 5 - protein, 328 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRADFGPRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTICDFLRGATVHRILGQQVPYATKGNQWVGYDDQESVKSKVQYLKDRQLAGAMVWALDLDDFQGSFCGQDLRFPLTNAIKDALAAT

SEQ ID NO: 6 - protein, 322 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTICDFLRGATVHRILGQQVPYATKGNQWVGYDDQESVKSKVQYLKDRQLAGAMVWALDLDDFQGSFCGQDLRFPLTNAIKDALAAT

SEQ ID NO: 7 - protein, 270 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRADFGPRFSKIASNTQSRRTFIKSVPPFLRTHGFDGLDLAWLYPGRRDKQHFTTLIKEMKAEFIKEAQPGKKQLLLSAALSAGKVTIDSSYDIAKISQPPPFLLAPRLILTPPLMPVRNKEQPSSLISPESRAMGLGKPRSKEQRLFKMTSELSLGSQGGVNSVLN

SEQ ID NO: 8 - protein, 264 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQPPPFLLAPRLILTPPLMPVRNKEQPSSLISPESRAMGLGKPRSKEQRLFKMTSELSLGSQGGVNSVLN

SEQ ID NO: 9 - DNA, 1747 bp, Homo sapiens
  source 1..1747: mol_type = other DNA
gaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgtggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtg

SEQ ID NO: 10 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 11 - DNA, 741 bp, Homo sapiens
  source 1..741: mol_type = other DNA
gagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaa

SEQ ID NO: 12 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 13 - protein, 699 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 14 - protein, 493 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 15 - protein, 361 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 16 - protein, 139 aa, Homo sapiens
MDKFWWHAAWGLCLVPLSLAQIDLNITCRFAGVFHVEKNGRYSISRTEAADLCKAFNSTLPTMAQMEKALSIGFETCSLHCSQQSKKVWAEEKASDQQWQWSCGGQKAKWTQRRGQQVSGNGAFGEQGVVRNSRPVYDS

SEQ ID NO: 17 - DNA, 5431 bp, Homo sapiens
  source 1..5431: mol_type = other DNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 18 - DNA, 5302 bp, Homo sapiens
  source 1..5302: mol_type = other DNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 19 - DNA, 4684 bp, Homo sapiens
  source 1..4684: mol_type = other DNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 20 - DNA, 4288 bp, Homo sapiens
  source 1..4288: mol_type = other DNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 21 - DNA, 3668 bp, Homo sapiens
  source 1..3668: mol_type = other DNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 22 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 23 - protein, 374 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 24 - protein, 322 aa, Homo sapiens
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

SEQ ID NO: 25 - protein, 724 aa, Homo sapiens
MSFPKAPLKRFNDPSGCAPSPGAYDVKTLEVLKGPVSFQKSQRFKQQKESKQNLNVDKDTTLPASARKVKSSESKESQKNDKDLKILEKEIRVLLQERGAQDRRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEENIVILSKQVEDLNVKCQLLEKEKEDHVNRNREHNENLNAEMQNLKQKFILEQQEREKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 26 - protein, 709 aa, Homo sapiens
MSFPKAPLKRFNDPSGCAPSPGAYDVKTLEVLKGPVSFQKSQRFKQQKESKQNLNVDKDTTLPASARKVKSSESKIRVLLQERGAQDRRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEENIVILSKQVEDLNVKCQLLEKEKEDHVNRNREHNENLNAEMQNLKQKFILEQQEREKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 27 - protein, 577 aa, Homo sapiens
MRQDAPKPTPAACRCSGLARRVLTIAFALLILGLMTWAYAAGVPLASDRYGLLAFGLYGAFLSAHLVAQSLFAYLEHRRVAAAARGPLDAATARSVALTISAYQEDPAYLRQCLASARALLYPRARLRVLMVVDGNRAEDLYMVDMFREVFADEDPATYVWDGNYHQPWEPAAAGAVGAGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCLRMVLLSLYAPLYMCGLLPAKFLALVTMNQSGWGTSGRRKLAANYVPLLPLALWALLLLGGLVRSVAHEARADWSGPSRAAEAYHLAAGAGAYVGYWVAMLTLYWVGVRRLCRRRTGGYRVQV

SEQ ID NO: 28 - protein, 578 aa, Homo sapiens
MRQQDAPKPTPAACRCSGLARRVLTIAFALLILGLMTWAYAAGVPLASDRYGLLAFGLYGAFLSAHLVAQSLFAYLEHRRVAAAARGPLDAATARSVALTISAYQEDPAYLRQCLASARALLYPRARLRVLMVVDGNRAEDLYMVDMFREVFADEDPATYVWDGNYHQPWEPAAAGAVGAGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCLRMVLLSLYAPLYMCGLLPAKFLALVTMNQSGWGTSGRRKLAANYVPLLPLALWALLLLGGLVRSVAHEARADWSGPSRAAEAYHLAAGAGAYVGYWVAMLTLYWVGVRRLCRRRTGGYRVQV

SEQ ID NO: 29 - protein, 552 aa, Homo sapiens
MHCERFLCILRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSEDDLYMMDIFSEVMGRDKSATYIWKNNFHEKGPGETDESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAIITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKRPFSESKQTVLIVGTLLYACYWVMLLTLYVVLINKCGRRKKGQQYDMVLDV

SEQ ID NO: 30 - protein, 553 aa, Homo sapiens
MPVQLTTALRVVGTSLFALAVLGGILAAYVTGYQFIHTEKHYLSFGLYGAILGLHLLIQSLFAFLEHRRMRRAGQALKLPSPRRGSVALCIAAYQEDPDYLRKCLRSAQRISFPDLKVVMVVDGNRQEDAYMLDIFHEVLGGTEQAGFFVWRSNFHEAGEGETEASLQEGMDRVRDVVRASTFSCIMQKWGGKREVMYTAFKALGDSVDYIQVCDSDTVLDPACTIEMLRVLEEDPQVGGVGGDVQILNKYDSWISFLSSVRYWMAFNVERACQSYFGCVQCISGPLGMYRNSLLQQFLEDWYHQKFLGSKCSFGDDRHLTNRVLSLGYRTKYTARSKCLTETPTKYLRWLNQQTRWSKSYFREWLYNSLWFHKHHLWMTYESVVTGFFPFFLIATVIQLFYRGRIWNILLFLLTVQLVGIIKATYACFLRGNAEMIFMSLYSLLYMSSLLPAKIFAIATINKSGWGTSGRKTIVVNFIGLIPVSIWVAVLLGGLAYTAYCQDLFSETELAFLVSGAILYGCYWVALLMLYLAIIARRCGKKPEQYSLAFAEV

SEQ ID NO: 31 - protein, 281 aa, Homo sapiens
MPVQLTTALRVVGTSLFALAVLGGILAAYVTGYQFIHTEKHYLSFGLYGAILGLHLLIQSLFAFLEHRRMRRAGQALKLPSPRRGSVALCIAAYQEDPDYLRKCLRSAQRISFPDLKVVMVVDGNRQEDAYMLDIFHEVLGGTEQAGFFVWRSNFHEAGEGETEASLQEGMDRVRDVVRASTFSCIMQKWGGKREVMYTAFKALGDSVDYIQVCDSDTVLDPACTIEMLRVLEEDPQVGGVGGDVQPPGKGMAVEDDQVQAAQVRATEAWSVHQRHVSREQ

SEQ ID NO: 32 - DNA, 2107 bp, Homo sapiens
  source 1..2107: mol_type = other DNA
acagagaggctgggagagcaagccggagagaagagagagcccggccagacccactgcgatgagacaggacgcgcccaagcccactcctgcagcctgccgctgctccggcctggcccggagggtgctgaccatcgccttcgccctgctcatcctgggcctcatgacctgggcctacgccgccggggtgccgctggcctccgatcgctacggcctcctggccttcggcctctacggggccttcctttcagcgcacctggtggcgcagagcctcttcgcgtacctggagcaccggcgggtggcggcggcggcgcgggggccgctggatgcagccaccgcgcgcagtgtggcgctgaccatctccgcctaccaggaggaccccgcgtacctgcgccagtgcctggcgtccgcccgcgccctgctgtacccgcgcgcgcggctgcgcgtcctcatggtggtggatggcaaccgcgccgaggacctctacatggtcgacatgttccgcgaggtcttcgctgacgaggaccccgccacgtacgtgtgggacggcaactaccaccagccctgggaacccgcggcggcgggcgcggtgggcgccggagcctatcgggaggtggaggcggaggatcctgggcggctggcagtggaggcgctggtgaggactcgcaggtgcgtgtgcgtggcgcagcgctggggcggcaagcgcgaggtcatgtacacagccttcaaggcgctcggagattcggtggactacgtgcaggtctgtgactcggacacaaggttggaccccatggcactgctggagctcgtgcgggtactggacgaggacccccgggtaggggctgttggtggggacgtgcggatccttaaccctctggactcctgggtcagcttcctaagcagcctgcgatactgggtagccttcaatgtggagcgggcttgtcagagctacttccactgtgtatcctgcatcagcggtcctctaggcctatataggaataacctcttgcagcagtttcttgaggcctggtacaaccagaagttcctgggtacccactgtacttttggggatgaccggcacctcaccaaccgcatgctcagcatgggttatgctaccaagtacacctccaggtcccgctgctactcagagacgccctcgtccttcctgcggtggctgagccagcagacacgctggtccaagtcgtacttccgtgagtggctgtacaacgcgctctggtggcaccggcaccatgcgtggatgacctacgaggcggtggtctccggcctgttccccttcttcgtggcggccactgtgctgcgtctgttctacgcgggccgcccttgggcgctgctgtgggtgctgctgtgcgtgcagggcgtggcactggccaaggcggccttcgcggcctggctgcggggctgcctgcgcatggtgcttctgtcgctctacgcgcccctctacatgtgtggcctcctgcctgccaagttcctggcgctagtcaccatgaaccagagtggctggggcacctcgggccggcggaagctggccgctaactacgtccctctgctgcccctggcgctctgggcgctgctgctgcttgggggcctggtccgcagcgtagcacacgaggccagggccgactggagcggcccttcccgcgcagccgaggcctaccacttggccgcgggggccggcgcctacgtgggctactgggtggccatgttgacgctgtactgggtgggcgtgcggaggctttgccggcggcggaccgggggctaccgcgtccaggtgtgagtccagccacgcggatgccgcctcaagggtcttcaggggaggccagaggagagctgctgggccccgagccacgaacttgctgggtggttctctgggcctcagtttccctcctctgcaaaacgagggggtcagcccaagattcttcagtctggactatattgggactgggacttctgggtctccagggagggtatttattggtcagggtgtgggatctgaggagtggagggaaagggtcctgctttctcctcgttcttatttaatctccatttctactgtgtgatcaggatgtaataaagaattttatttattttc

SEQ ID NO: 33 - DNA, 2110 bp, Homo sapiens
  source 1..2110: mol_type = other DNA
acagagaggctgggagagcaagccggagagaagagagagcccggccagacccactgcgatgagacagcaggacgcgcccaagcccactcctgcagcctgccgctgctccggcctggcccggagggtgctgaccatcgccttcgccctgctcatcctgggcctcatgacctgggcctacgccgccggggtgccgctggcctccgatcgctacggcctcctggccttcggcctctacggggccttcctttcagcgcacctggtggcgcagagcctcttcgcgtacctggagcaccggcgggtggcggcggcggcgcgggggccgctggatgcagccaccgcgcgcagtgtggcgctgaccatctccgcctaccaggaggaccccgcgtacctgcgccagtgcctggcgtccgcccgcgccctgctgtacccgcgcgcgcggctgcgcgtcctcatggtggtggatggcaaccgcgccgaggacctctacatggtcgacatgttccgcgaggtcttcgctgacgaggaccccgccacgtacgtgtgggacggcaactaccaccagccctgggaacccgcggcggcgggcgcggtgggcgccggagcctatcgggaggtggaggcggaggatcctgggcggctggcagtggaggcgctggtgaggactcgcaggtgcgtgtgcgtggcgcagcgctggggcggcaagcgcgaggtcatgtacacagccttcaaggcgctcggagattcggtggactacgtgcaggtctgtgactcggacacaaggttggaccccatggcactgctggagctcgtgcgggtactggacgaggacccccgggtaggggctgttggtggggacgtgcggatccttaaccctctggactcctgggtcagcttcctaagcagcctgcgatactgggtagccttcaatgtggagcgggcttgtcagagctacttccactgtgtatcctgcatcagcggtcctctaggcctatataggaataacctcttgcagcagtttcttgaggcctggtacaaccagaagttcctgggtacccactgtacttttggggatgaccggcacctcaccaaccgcatgctcagcatgggttatgctaccaagtacacctccaggtcccgctgctactcagagacgccctcgtccttcctgcggtggctgagccagcagacacgctggtccaagtcgtacttccgtgagtggctgtacaacgcgctctggtggcaccggcaccatgcgtggatgacctacgaggcggtggtctccggcctgttccccttcttcgtggcggccactgtgctgcgtctgttctacgcgggccgcccttgggcgctgctgtgggtgctgctgtgcgtgcagggcgtggcactggccaaggcggccttcgcggcctggctgcggggctgcctgcgcatggtgcttctgtcgctctacgcgcccctctacatgtgtggcctcctgcctgccaagttcctggcgctagtcaccatgaaccagagtggctggggcacctcgggccggcggaagctggccgctaactacgtccctctgctgcccctggcgctctgggcgctgctgctgcttgggggcctggtccgcagcgtagcacacgaggccagggccgactggagcggcccttcccgcgcagccgaggcctaccacttggccgcgggggccggcgcctacgtgggctactgggtggccatgttgacgctgtactgggtgggcgtgcggaggctttgccggcggcggaccgggggctaccgcgtccaggtgtgagtccagccacgcggatgccgcctcaagggtcttcaggggaggccagaggagagctgctgggccccgagccacgaacttgctgggtggttctctgggcctcagtttccctcctctgcaaaacgagggggtcagcccaagattcttcagtctggactatattgggactgggacttctgggtctccagggagggtatttattggtcagggtgtgggatctgaggagtggagggaaagggtcctgctttctcctcgttcttatttaatctccatttctactgtgtgatcaggatgtaataaagaattttatttattttc

SEQ ID NO: 34 - DNA, 5860 bp, Homo sapiens
  source 1..5860: mol_type = other DNA
acagagaggctgggagagcaagccggagagaagagagagcccggccagacccactgcgatgagacagcaggacgcgcccaagcccactcctgcagcctgccgctgctccggcctggcccggagggtgctgaccatcgccttcgccctgctcatcctgggcctcatgacctgggcctacgccgccggggtgccgctggcctccgatcgctacggcctcctggccttcggcctctacggggccttcctttcagcgcacctggtggcgcagagcctcttcgcgtacctggagcaccggcgggtggcggcggcggcgcgggggccgctggatgcagccaccgcgcgcagtgtggcgctgaccatctccgcctaccaggaggaccccgcgtacctgcgccagtgcctggcgtccgcccgcgccctgctgtacccgcgcgcgcggctgcgcgtcctcatggtggtggatggcaaccgcgccgaggacctctacatggtcgacatgttccgcgaggtcttcgctgacgaggaccccgccacgtacgtgtgggacggcaactaccaccagccctgggaacccgcggcggcgggcgcggtgggcgccggagcctatcgggaggtggaggcggaggatcctgggcggctggcagtggaggcgctggtgaggactcgcaggtgcgtgtgcgtggcgcagcgctggggcggcaagcgcgaggtcatgtacacagccttcaaggcgctcggagattcggtggactacgtgcaggtctgtgactcggacacaaggttggaccccatggcactgctggagctcgtgcgggtactggacgaggacccccgggtaggggctgttggtggggacgtgcggatccttaaccctctggactcctgggtcagcttcctaagcagcctgcgatactgggtagccttcaatgtggagcgggcttgtcagagctacttccactgtgtatcctgcatcagcggtcctctaggcctatataggaataacctcttgcagcagtttcttgaggcctggtacaaccagaagttcctgggtacccactgtacttttggggatgaccggcacctcaccaaccgcatgctcagcatgggttatgctaccaagtacacctccaggtcccgctgctactcagagacgccctcgtccttcctgcggtggctgagccagcagacacgctggtccaagtcgtacttccgtgagtggctgtacaacgcgctctggtggcaccggcaccatgcgtggatgacctacgaggcggtggtctccggcctgttccccttcttcgtggcggccactgtgctgcgtctgttctacgcgggccgcccttgggcgctgctgtgggtgctgctgtgcgtgcagggcgtggcactggccaaggcggccttcgcggcctggctgcggggctgcctgcgcatggtgcttctgtcgctctacgcgcccctctacatgtgtggcctcctgcctgccaagttcctggcgctagtcaccatgaaccagagtggctggggcacctcgggccggcggaagctggccgctaactacgtccctctgctgcccctggcgctctgggcgctgctgctgcttgggggcctggtccgcagcgtagacccccttaagttggaggaggcagaagggcaacaacggcggggaaggagaagtcaagacgtctggaaagaattacccagtcctggcttcgagcagcccattgaaccagagacttgaaacagccccagccaaagacttttctcccaattctgcgcttcctgggttctgctgagtcttccacaggctttttttttttttttttttttttttaagacgaaaaagagattttctgttatcgggggcagaaagactgaagcccaaaaaaaaaaaaaaaaaaaaaagaaaagaaaagaaaaaagaaaagttaatttatttttaaagcataatttttttaagaattagactgaagtgcaacggaaacataaagagaatattagtgaaattattttttaaagtggggaagaatcaaacatttaagactcccctatcctttttaaatgttgtttttaaatttcttattttttttggccggtcgtctcaaattcatctgatctcttattacctcaattttggaaactgcccgccaccgaccctccgggaccacacagacaggctgaggacgactttatgaccaagagctgaacaagatgcattgtgagaggtttctatgtatcctgagaataattggaaccacactctttggagtctctctcctccttggaatcacagctgcttatattgttggctaccagtttatccaaacggataattactatttctcttttggactgtatggtgcctttttggcatcacacctcatcatccaaagcctgtttgcctttttggagcaccgaaaaatgaaaaaatccctagaaacccccataaagttgaacaaaacagttgccctttgcatcgctgcctatcaagaagatccagactacttaaggaaatgtttgcaatctgtgaaaaggctaacctaccctgggattaaagttgtcatggtcatagatgggaactcagaagatgacctttacatgatggacatcttcagtgaagtcatgggcagagacaaatcagccacttatatctggaagaacaacttccacgaaaagggtcccggtgagacagatgagtcacataaagaaagctcgcaacacgtaacgcaattggtcttgtccaacaaaagtatctgcatcatgcaaaaatggggtggaaaaagagaagtcatgtacacagccttcagagcactgggacgaagtgtggattatgtacaggtttgtgattcagacactatgcttgacccagcctcatctgtggagatggtaaaagttttagaagaagatcccatggttggaggtgttgggggagatgtccagattttaaacaagtacgattcctggatctcattcctcagcagtgtaagatattggatggcttttaatatagaaagggcctgtcagtcttattttgggtgtgttcagtgcattagtggacctctgggaatgtacagaaactccttgttgcatgagtttgtggaagattggtacaatcaagaatttatgggcaaccaatgtagctttggtgatgacaggcatctcacgaaccgggtgctgagcctgggctatgcaacaaaatacacagctcgatctaagtgccttactgaaacacctatagaatatctcagatggctaaaccagcagacccgttggagcaagtcctacttccgagaatggctgtacaatgcaatgtggtttcacaaacatcacttgtggatgacctacgaagcgattatcactggattctttcctttctttctcattgccacagtaatccagctcttctaccggggtaaaatttggaacattctcctcttcttgttaactgtccagctagtaggtctcataaaatcatcttttgccagctgccttagaggaaatatcgtcatggtcttcatgtctctctactcagtgttatacatgtcgagtttacttcccgccaagatgtttgcaattgcaacaataaacaaagctgggtggggcacatcaggaaggaaaaccattgttgttaatttcataggactcattccagtatcagtttggtttacaatcctcctgggtggtgtgattttcaccatttataaggagtctaaaaggccattttcagaatccaaacagacagttctaattgttggaacgttgctctatgcatgctattgggtcatgcttttgacgctgtatgtagttctcatcaataagtgtggcaggcggaagaagggacaacaatatgacatggtgcttgatgtatgatcttccatgttttgacgtttgcagtcacacacaacaccttagttcctctaggggctgtacagtattgtggcatcagataatgccaccaaaggagacatatcactgctgctgggacttgaacaaagacatttatatgggtttattttcattctgccaaagtaaaacaatacatcaacaagaagaaactcagatttaacctgttatttctatgaaaatgggatgaattctttgtttatgcactttttccttactgtgcatccgcctgaaagtgttttgccctatatacctcactagccatgctttatgtgggttatcatggaagaaaaggattttggaaactcaaggaaaagttctttcaacctatacaacctaacttatggactgttttgatagatgataattttttttttttaggaaggattttctttttaactttaccaaatgaaatgccaaaggaagttttaaaggccgttggctgtgctgtattttgatataattgtactgtgtttttaaattttgtatgccaatcttaaagacaaattttgcatattctctattttacttttctgccaaaataaacctgttcttccttttttaaaataaaataagttcttaaaaaatttatacttaaaaaatcctgcccaaaatgtgaagcttggttgactgatgttcatgatagaaagaataaaatgtttctctctctctaccttttaaaattgaatagtttatttctgtgaaagaagtatttaaactttcaatattttaactttttgtttttatttcttttagaaaaggccaatatacctatcacactttggaagtaaaaatacacactttcgtgtgtacctaaaaaaaaaatcgttgaaaatcaaggccaaaggtagtgcaattttttcattaagatttaaaaaaaagggaatgatagtctttgaaagaaaacagtaggcatccagcactggacaaaacatgggtatcaaagatgaataatctttggagattctggcagtgttttcccagaacaagtcaagtggaaagtggagaaattatctgtataattttggacacatacaatgcagtttatcaaaggttttgttctgtggcctgaatttactgggtcctacctacacattgaacatgttttgcctgtcttttttttttttttcaacttgccagttcactttacatgttagtataatgtttacacgggtgagttggataaattataaaacatataaattcaaaattggcagatagaatcaccaagtatctatcctcttttactttcaaatgaggaattttgtttttctgaattacacagatcatcacttcctatttcctgttctggacctgtataaaaatgtctacacagtagaagtgacatcaaggtttaataagtatatcaatgattggcacatataaaaattgttgaaccacatactctgaacttggctaatttagttactgcaaggcctccattatccagttttattttttacacgattgaccttgccttgtagctggtgctgtgtagacctgtgttgaaaacacaatcggaatatatgaataattgaataaacagcattatggtgaggcagagacacatggagaagtgttaaaaaaaaaatgggcttcctgcctttctgcctcttttttatgcagtcatctatgttacatctatcctgcctaagaaaaagctgcacatcctaccttcagagtacaaaaaggtacatctgagactcaagactctcactgattggagagcttgtggaaaacaaaacacaccatgccaataaatgagatgaaaacttgagtttgcctttttaactatttatgttctaagttaagctttgataacattcaaatgtcaaattctctcattcttataaaaagttgaattaattgcctgtatttattttagcaattattcaatgtatttccagtataggatgtatagtataattaattttttgtaaataaaatatttttgataaga

SEQ ID NO: 35 - DNA, 4191 bp, Homo sapiens
  source 1..4191: mol_type = other DNA
aattctcttcagcgcctctttcctgccagcaggaaggttttgctgccttggctttcgggagccccctagacagcgaactgccccttcgcgtgcagaagtcggggaagagtgctctcagctcgccttccttgcaggatgccggtgcagctgacgacagccctgcgtgtggtgggcaccagcctgtttgccctggcagtgctgggtggcatcctggcagcctatgtgacgggctaccagttcatccacacggaaaagcactacctgtccttcggcctgtacggcgccatcctgggcctgcacctgctcattcagagcctttttgccttcctggagcaccggcgcatgcgacgtgccggccaggccctgaagctgccctccccgcggcggggctcggtggcactgtgcattgccgcataccaggaggaccctgactacttgcgcaagtgcctgcgctcggcccagcgcatctccttccctgacctcaaggtggtcatggtggtggatggcaaccgccaggaggacgcctacatgctggacatcttccacgaggtgctgggcggcaccgagcaggccggcttctttgtgtggcgcagcaacttccatgaggcaggcgagggtgagacggaggccagcctgcaggagggcatggaccgtgtgcgggatgtggtgcgggccagcaccttctcgtgcatcatgcagaagtggggaggcaagcgcgaggtcatgtacacggccttcaaggccctcggcgattcggtggactacatccaggtgtgcgactctgacactgtgctggatccagcctgcaccatcgagatgcttcgagtcctggaggaggatccccaagtagggggagtcgggggagatgtccagatcctcaacaagtacgactcatggatttccttcctgagcagcgtgcggtactggatggccttcaacgtggagcgggcctgccagtcctactttggctgtgtgcagtgtattagtgggcccttgggcatgtaccgcaacagcctcctccagcagttcctggaggactggtaccatcagaagttcctaggcagcaagtgcagcttcggggatgaccggcacctcaccaaccgagtcctgagccttggctaccgaactaagtataccgcgcgctccaagtgcctcacagagacccccactaagtacctccggtggctcaaccagcaaacccgctggagcaagtcttacttccgggagtggctctacaactctctgtggttccataagcaccacctctggatgacctacgagtcagtggtcacgggtttcttccccttcttcctcattgccacggttatacagcttttctaccggggccgcatctggaacattctcctcttcctgctgacggtgcagctggtgggcattatcaaggccacctacgcctgcttccttcggggcaatgcagagatgatcttcatgtccctctactccctcctctatatgtccagccttctgccggccaagatctttgccattgctaccatcaacaaatctggctggggcacctctggccgaaaaaccattgtggtgaacttcattggcctcattcctgtgtccatctgggtggcagttctcctgggagggctggcctacacagcttattgccaggacctgttcagtgagacagagctagccttccttgtctctggggctatactgtatggctgctactgggtggccctcctcatgctatatctggccatcatcgcccggcgatgtgggaagaagccggagcagtacagcttggcttttgctgaggtgtgacatggcccccaagcagagcgggtaaagtgcaatgggtaagggagggaaggggaatggaagagaaaagacagggtgggagggaggagggagtgctgtgttttagtctcttaatggtccaaaggacaaatctaaaatgcaaagaacggtgatgtagtatggcctgacagctctgtttagaggaggcaacactgatcccccagatgcagggctgcaggggattctgtgttttcagactgcctgtctgcttgcatctgcacataggcagtagcctcctcctgggctccagagggcactcagaagttgtgctaaaccaagttaagtcccattcagtggcaacttgtgataggtacctgagtgacggcaacctgcggaaggaggttctcccagcccatctgaacacaaccagaggtggcaggagaatttctactgagcgagctgggccggttagtgtatgtcacccccaccccacccctaagtagtcatcaatgcaataagattgcgcctgagatacaaggcccagaagcctgatctttgggcatcagaaaacagggtccaggaatggtgctttatgtgagataccccactccacatcaacattccagggatgagccaaaccagcagggagttagcactgaactgcttttaaaagtgcacattaaaaaggaaagtttgccaggaggaacaaagagattgtggtggtgctaaaggaggccataagctacacagaggccttgggtgttccacctggaaactgctcagacgtctagatgggttcttagcttgtctgtgatctctgctggggagataaaaagattaagccccaacatgttcagaaaagaagtgaagtcttgggtattttaacctgtatactcttgaattcctctcaaattcagctctgatctgaggctaagacacactccccacttcactttcttcaaagccacattttttgaggtatcactgcagtcacctcttctaccctcatcatcataggtaaggttttcaaggtggcaattggggcggagccccggctcttatagaagcttcagcaggaggcaagcgtgttctcagcacatatgggaactatgaggagcctctgatcaaattggctacaatcttggagctgcttggacggattccttggcagccgggttagcatgtgtgactttcaggctactgttcttgacaatcatctccaatggaaagcttttcagtgttcccaaagtgaactctcaaatccaaaatggttatctttgagaccatccattctcctcagtggcttctccagggaattcttacagccaagttgtgacagtcactgcatttgcctgcttctttccagaaaccaaactaggagatgaaactggttcctacatcctaaggttcttgctttctctctcatgcctcctgaggctgtttttggctgttttccctctgctgcttttggggaatgaggggaagccattttccagtgacttgcaatccaggctgttctcagcgttttgagtttaaaacctgggatcctgactaagcctttgacttaagggttgcttgcttgccctccaaatgtcctttctcagaggggccagctaacccgtgcagaaccagcactaaggtggacagcagacaagagggcaagcctctagtgtaccaagtgcttcctacaaagacgcaaggtgtgctctgagccacagatgggcaaaccctggtgctttccttcatctcccacgaactcaagggttttccaggtgtagctaacagttgccacatcacacagacctcgagtttctggtaagactgctggttgacatcagacccaacccatgaaggctggaaggcagcaggcatttgctaaggcagctgatccaggcaatcgttctgctggccaagaagttaaactattttgagcattagaatggaggaaatccggtcagccaagtgcagagttcagacttcgctaagggcttgtttttcttcagcatttacttgaagattaatgtaggatgacaggctctcctggctgtcctaccatcagctctgccttgcactgtggtcgtcaactttcctcaaatcaaaaacaggcaggtacaggtagtgggctcacaacgtttgacctcgactggtttttctaagttattttgtacatttttcagcagcaaaaccaaactgggtcttcagctttatccccgtttcttgcaagggaagagcctttatacaattggacgcattttggtttttcctcattgagaattcaaatcctcttttgtattgtttctacaataatttgtaaacatatttatttttacctgcttttttttttttttttaattttcaggtcaagttttttatactgcacttatttgtcaaaataaagattctcacatatgccggtta

SEQ ID NO: 36 - DNA, 4212 bp, Homo sapiens
  source 1..4212: mol_type = other DNA
gagtgcgttcgcggctgctttgacctggtgggcgccgcctccggcactgcaccgaggcggggcgccagcgcccaggttgctgggctggccttggcgcccccttcccctacccagagcgcaggcaggcaggggggtcccgccgcgccccttcagcatatgccggtgcagctgacgacagccctgcgtgtggtgggcaccagcctgtttgccctggcagtgctgggtggcatcctggcagcctatgtgacgggctaccagttcatccacacggaaaagcactacctgtccttcggcctgtacggcgccatcctgggcctgcacctgctcattcagagcctttttgccttcctggagcaccggcgcatgcgacgtgccggccaggccctgaagctgccctccccgcggcggggctcggtggcactgtgcattgccgcataccaggaggaccctgactacttgcgcaagtgcctgcgctcggcccagcgcatctccttccctgacctcaaggtggtcatggtggtggatggcaaccgccaggaggacgcctacatgctggacatcttccacgaggtgctgggcggcaccgagcaggccggcttctttgtgtggcgcagcaacttccatgaggcaggcgagggtgagacggaggccagcctgcaggagggcatggaccgtgtgcgggatgtggtgcgggccagcaccttctcgtgcatcatgcagaagtggggaggcaagcgcgaggtcatgtacacggccttcaaggccctcggcgattcggtggactacatccaggtgtgcgactctgacactgtgctggatccagcctgcaccatcgagatgcttcgagtcctggaggaggatccccaagtagggggagtcgggggagatgtccagatcctcaacaagtacgactcatggatttccttcctgagcagcgtgcggtactggatggccttcaacgtggagcgggcctgccagtcctactttggctgtgtgcagtgtattagtgggcccttgggcatgtaccgcaacagcctcctccagcagttcctggaggactggtaccatcagaagttcctaggcagcaagtgcagcttcggggatgaccggcacctcaccaaccgagtcctgagccttggctaccgaactaagtataccgcgcgctccaagtgcctcacagagacccccactaagtacctccggtggctcaaccagcaaacccgctggagcaagtcttacttccgggagtggctctacaactctctgtggttccataagcaccacctctggatgacctacgagtcagtggtcacgggtttcttccccttcttcctcattgccacggttatacagcttttctaccggggccgcatctggaacattctcctcttcctgctgacggtgcagctggtgggcattatcaaggccacctacgcctgcttccttcggggcaatgcagagatgatcttcatgtccctctactccctcctctatatgtccagccttctgccggccaagatctttgccattgctaccatcaacaaatctggctggggcacctctggccgaaaaaccattgtggtgaacttcattggcctcattcctgtgtccatctgggtggcagttctcctgggagggctggcctacacagcttattgccaggacctgttcagtgagacagagctagccttccttgtctctggggctatactgtatggctgctactgggtggccctcctcatgctatatctggccatcatcgcccggcgatgtgggaagaagccggagcagtacagcttggcttttgctgaggtgtgacatggcccccaagcagagcgggtaaagtgcaatgggtaagggagggaaggggaatggaagagaaaagacagggtgggagggaggagggagtgctgtgttttagtctcttaatggtccaaaggacaaatctaaaatgcaaagaacggtgatgtagtatggcctgacagctctgtttagaggaggcaacactgatcccccagatgcagggctgcaggggattctgtgttttcagactgcctgtctgcttgcatctgcacataggcagtagcctcctcctgggctccagagggcactcagaagttgtgctaaaccaagttaagtcccattcagtggcaacttgtgataggtacctgagtgacggcaacctgcggaaggaggttctcccagcccatctgaacacaaccagaggtggcaggagaatttctactgagcgagctgggccggttagtgtatgtcacccccaccccacccctaagtagtcatcaatgcaataagattgcgcctgagatacaaggcccagaagcctgatctttgggcatcagaaaacagggtccaggaatggtgctttatgtgagataccccactccacatcaacattccagggatgagccaaaccagcagggagttagcactgaactgcttttaaaagtgcacattaaaaaggaaagtttgccaggaggaacaaagagattgtggtggtgctaaaggaggccataagctacacagaggccttgggtgttccacctggaaactgctcagacgtctagatgggttcttagcttgtctgtgatctctgctggggagataaaaagattaagccccaacatgttcagaaaagaagtgaagtcttgggtattttaacctgtatactcttgaattcctctcaaattcagctctgatctgaggctaagacacactccccacttcactttcttcaaagccacattttttgaggtatcactgcagtcacctcttctaccctcatcatcataggtaaggttttcaaggtggcaattggggcggagccccggctcttatagaagcttcagcaggaggcaagcgtgttctcagcacatatgggaactatgaggagcctctgatcaaattggctacaatcttggagctgcttggacggattccttggcagccgggttagcatgtgtgactttcaggctactgttcttgacaatcatctccaatggaaagcttttcagtgttcccaaagtgaactctcaaatccaaaatggttatctttgagaccatccattctcctcagtggcttctccagggaattcttacagccaagttgtgacagtcactgcatttgcctgcttctttccagaaaccaaactaggagatgaaactggttcctacatcctaaggttcttgctttctctctcatgcctcctgaggctgtttttggctgttttccctctgctgcttttggggaatgaggggaagccattttccagtgacttgcaatccaggctgttctcagcgttttgagtttaaaacctgggatcctgactaagcctttgacttaagggttgcttgcttgccctccaaatgtcctttctcagaggggccagctaacccgtgcagaaccagcactaaggtggacagcagacaagagggcaagcctctagtgtaccaagtgcttcctacaaagacgcaaggtgtgctctgagccacagatgggcaaaccctggtgctttccttcatctcccacgaactcaagggttttccaggtgtagctaacagttgccacatcacacagacctcgagtttctggtaagactgctggttgacatcagacccaacccatgaaggctggaaggcagcaggcatttgctaaggcagctgatccaggcaatcgttctgctggccaagaagttaaactattttgagcattagaatggaggaaatccggtcagccaagtgcagagttcagacttcgctaagggcttgtttttcttcagcatttacttgaagattaatgtaggatgacaggctctcctggctgtcctaccatcagctctgccttgcactgtggtcgtcaactttcctcaaatcaaaaacaggcaggtacaggtagtgggctcacaacgtttgacctcgactggtttttctaagttattttgtacatttttcagcagcaaaaccaaactgggtcttcagctttatccccgtttcttgcaagggaagagcctttatacaattggacgcattttggtttttcctcattgagaattcaaatcctcttttgtattgtttctacaataatttgtaaacatatttatttttacctgcttttttttttttttttaattttcaggtcaagttttttatactgcacttatttgtcaaaataaagattctcacatatgccggtta

SEQ ID NO: 37 - DNA, 1198 bp, Homo sapiens
  source 1..1198: mol_type = other DNA
agtcggagcgcgcggcggcgcggagcggagcgggaggaggggcatggagccgccgcgggcctgctgagctccggagcgcggcagccggcggcacgatgccggtgcagctgacgacagccctgcgtgtggtgggcaccagcctgtttgccctggcagtgctgggtggcatcctggcagcctatgtgacgggctaccagttcatccacacggaaaagcactacctgtccttcggcctgtacggcgccatcctgggcctgcacctgctcattcagagcctttttgccttcctggagcaccggcgcatgcgacgtgccggccaggccctgaagctgccctccccgcggcggggctcggtggcactgtgcattgccgcataccaggaggaccctgactacttgcgcaagtgcctgcgctcggcccagcgcatctccttccctgacctcaaggtggtcatggtggtggatggcaaccgccaggaggacgcctacatgctggacatcttccacgaggtgctgggcggcaccgagcaggccggcttctttgtgtggcgcagcaacttccatgaggcaggcgagggtgagacggaggccagcctgcaggagggcatggaccgtgtgcgggatgtggtgcgggccagcaccttctcgtgcatcatgcagaagtggggaggcaagcgcgaggtcatgtacacggccttcaaggccctcggcgattcggtggactacatccaggtgtgcgactctgacactgtgctggatccagcctgcaccatcgagatgcttcgagtcctggaggaggatccccaagtagggggagtcgggggagatgtccagcccccagggaaaggtatggcagtagaggatgaccaggtccaagctgcccaggtcagagctacggaagcatggtccgttcaccaacgccacgtttctagagagcagtgagctgattctccaatggtgagcaggggactacatgtgaactgggacctgcaggccaatgtatccctgaggaaaagtccacaagaacaattcaagaaactagtaagaaacaatgggcagaaagctgtgggacgaaagcacagcaggcttctgggaggctggagatcctttctctcaagggcaggattattcccacctgccacagttcacatgccacaaataacatctcaccttcagtcaagaaaaacgcaaaggaaaaa

SEQ ID NO: 38 - protein, 451 aa, Homo sapiens
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 39 - protein, 219 aa, Homo sapiens
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 446 aa, Homo sapiens
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTYLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, Homo sapiens
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 449 aa, Homo sapiens
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 213 aa, Homo sapiens
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 449 aa, Homo sapiens
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 215 aa, Homo sapiens
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 453 aa, Homo sapiens
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 216 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 461 aa, Homo sapiens
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 219 aa, Homo sapiens
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC